(19) United States
(12) Patent Application Publication
(10) Pub. No.: US 2024/0066102 A1
(43) Pub. Date: Feb. 29, 2024

(54) Genome editing approaches to treat Spinal Muscular Atrophy

(71) Applicant: The General Hospital Corporation, Boston, MA (US)

(72) Inventors: Benjamin Kleinstiver, Boston, MA (US); Christiano Robles Alves, Cambridge, MA (US); Kathryn J. Swoboda, Cambridge, MA (US); Kathleen A. Christie, Boston, MA (US)

(21) Appl. No.: 18/271,190

(22) PCT Filed: Jan. 10, 2022
(86) PCT No.: PCT/US22/11823

§ 371 Date: Jul. 6, 2023

Related U.S. Application Data
(60) Provisional application No. 63/135,471, filed on Jan. 8, 2021.

(51) Int. Cl. A61K 38/46; C12N 15/86

(52) U.S. Cl. CPC A61K 38/465; C12N 15/86; C12N 2750/14133; C12N 2750/14143

(57) ABSTRACT

Described herein are methods and compositions for treating subjects with spinal muscular atrophy (SMA) using CRISPR editing of exon 7 and/or intron 7 of SMN2.

### CLAIM OF PRIORITY

This application claims the benefit of U.S. Provisional Patent Application Ser. No. 63/135,471, filed on Jan. 8, 2021. The entire contents of the foregoing are hereby incorporated by reference.

### TECHNICAL FIELD

Described herein are methods and compositions for treating subjects with spinal muscular atrophy (SMA) using CRISPR editing of exon 7 and/or intron 7 of SMN2.

### BACKGROUND

Spinal muscular atrophy (SMA) remains the leading genetic cause of infantile death worldwide. SMA is a devastating neuromuscular disease caused by the homozygous deletion (95% of cases) or compound heterozygous mutation (5% of cases) in the survival motor neuron 1 (SMN1) gene. The disease is divided into clinical subtypes based on age of onset and maximum achievement of motor abilities. The severe infantile type 1 variant is the most frequent subtype, accounting for more than 50% of incident cases. By the time such patients present with symptoms, severe denervation has already occurred, and the majority of patients with SMA type 1 die early in life.

There is presently no permanent cure for SMA.

### SUMMARY

Described herein is a strategy to edit the SMN2 gene to produce full-length SMN protein by applying CRISPR technologies. We are leveraging novel variants of Streptococcus pyogenes Cas9 (e.g., SpG and SpRY) that allow us to precisely target previously intractable complex genomic regions in the SMN2 gene. The recent development of base editors (fusion proteins that comprise a Cas9 nickase (nCas9) or catalytically inactive dead Cas9 (dCas9) fused to a deaminase domain), including adenine base editors with enhanced activities (ABE8e), allow efficient introduction of single A-to-G nucleotide changes at loci in living cells. These novel technologies have allowed us to develop an efficient approach to edit the C-to-T transition in the exon 7 of SMN2, which will correct its genetic defect and restore SMN protein expression. In principle, this will create a lasting single-dose cure for SMA.

Thus provided herein are base editors comprising a Cas9, wherein the Cas9 is a nickase or catalytically inactive (e.g., where the Cas9 is SpCas9 or based on Cas9, includes a D10A mutation), and a deaminase domain that modifies adenosine DNA bases; and one or more guide RNAs that target the base editor to deaminate the adenine at position 6 in exon 7 (position 6 of CTAAAACCCT (SEQ ID NO:1)) and/or to deaminate adenines within the ISS-N1 and ISS+100 motifs in SMN2 intron 7, and methods for treating a subject who has spinal muscular atrophy (SMA), by administering to the subject a therapeutically effective amount of the base editors and guide RNAs described herein. Also provided are base editors comprising a Cas9, wherein the Cas9 is a nickase or catalytically inactive, and a deaminase domain that modifies adenosine DNA bases; and a guide RNA (gRNA) that targets the base editor to deaminate the adenine at position 6 in SMN2 exon 7 (position 6 of CTAAAACCCT (SEQ ID NO:1) and/or to deaminate adenines within the ISS-N1 and ISS+100 motifs in SMN2 intron 7, for use in a method of treating a subject who has spinal muscular atrophy (SMA). Additionally provided are compositions comprising (i) a base editor comprising a Cas9, wherein the Cas9 is a nickase or catalytically inactive, and a deaminase domain that modifies adenosine DNA bases; and (ii) a guide RNA (gRNA) that targets the base editor to deaminate the adenine at position 6 in SMN2 exon 7 (position 6 of CTAAAACCCT (SEQ ID NO:1) and/or to deaminate adenines within the ISS-N1 and ISS+100 motifs in SMN2 intron 7. Other Cas proteins other than Cas9 can also be used.

In some embodiments, the Cas9 is wild-type SpCas9, or a SpCas9 variant that targets NGG, NGN, NRN, or NYN PAMs. In some embodiments, the variant is an SpCas9 variant that comprises A61R, L1111R, D1135L, S1136W, G1218K, E1219Q, N1317R, A1322R, R1333P, R1335Q, and T1337R substitutions (e.g. SpRY); SpRY that also comprises HF1 mutations N497A, R661A, Q695A, Q926A (e.g. SpRY-HF1); SpRY that also comprises a HiFi mutation R691A (e.g. SpRY-HiFi), SpCas9 that comprises D1135L, S1136W, G1218K, E1219Q, R1335Q, and T1337R substitutions (e.g. SpG); SpCas9 that comprises D10T, I322V, S409I, E427G, R654L, R753G, R1114G, D1135N, V1139A, D1180G, E1219V, Q1221H, A1320V, R1333K substitutions or SpCas9 that comprises R1114G, D1135N, V1139A, D1180G, E1219V, Q1221H, A1320V, R1333K substitutions (e.g. SpCas9-NRRH). The Cas9 also includes mutations that reduce or abrogate nuclease activity, e.g., D10A in SpCas9. In some embodiments, the adenosine deaminase domain is from ABE8e or ABE8.20-m. In some embodiments, the guide RNA comprises a sequence shown in Table 1, preferably SMN2-ex7-gRNA-A5, SMN2-ex7-gRNA-A7, SMN2-ex7-gRNA-A7_G, SMN2-ex7-gRNA-A8, SMN2-ex7-gRNA-A8_G, SMN2-ex7-gRNA-A10, SMN2-ex7-gRNA-A10_G, ISS-N1-gRNA1, ISS-N1-gRNA3, ISS+100-gRNA3, ISS+100-gRNA4, or ISS+100-gRNA6.

In some embodiments, the methods and/or compositions comprise administering a base editor and gRNA as shown in the following table:

| Base editor | gRNA |
|---|---|
| ABE8e-SpRY, ABE8e-SpRY- | SMN2-ex7-gRNA-A5, SMN2-ex7-gRNA-A5_G, SMN2- |
| HF1, or ABE8e-SpRY-HiFi | ex7-gRNA-A7, SMN2-ex7-gRNA-A7_G, SMN2-ex7- |
| | gRNA-A8, SMN2-ex7-gRNA-A8_G |
| ABE8e-SpCas9 | SMN2-ex7-gRNA-A10 (or SMN2-ex7-gRNA-A10_G) |
| ABE8e-SpRY | ISS-N1-gRNA1, ISS-N1-gRNA3 alone or in |
| | addition to SMN2-ex7-gRNA-A8 |
| ABE8e-SpRY | ISS + 100-gRNA4, ISS + 100-gRNA3, ISS + 100-gRNA7 |
| | alone or in addition to SMN2-ex7-gRNA-A8 |

In some embodiments, the base editor and gRNA are in, or are administered as, a ribonucleoprotein (RNP) complex. In some embodiments, the RNP complex is administered systemically (e.g. intravenously, intraperitoneally, etc.) or by intrathecal, intracerebroventricular, intracerebral, or other routes of injection or infusion.

In some embodiments, the methods include administering, or the compositions include, nucleic acids encoding the base editor and gRNA. In some embodiments, the nucleic acids comprise at least one viral vector comprising sequences encoding the base editor and gRNA. In some embodiments, the viral vector is an AAV. In some embodiments, provided herein are compositions comprising (i) mRNA encoding the base editor and (ii) one or more guide RNAs, preferably in lipid nanoparticles (LNPs), and methods of use thereof.

In some embodiments, the composition (e.g., comprising LNPs or RNPs) or viral vector is administered, or is formulated to be administered, systemically (e.g. intravenously, intraperitoneally, etc.) or by intrathecal, intracerebroventricular, intracerebral, or other routes of injection or infusion.

Also provided herein are the vectors encoding the base editors and/or gRNAs, and RNPs comprising the base editors and gRNAs, as described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. A schematic illustration of a complex of SpRY fused to a deaminase enzyme and gRNA, placed in the specific region to edit a single nucleotide.

FIGS. 2A-B. Correction of a C-to-T mutation in SMN2 to restore SMN protein expression. (A) Editable window in the SMN2 gene with target nucleotide and neighboring adenines

(top strand, 5′-3′ sequence:
CCTTTATTTTCCTTACAGGGTTTTAGACAAAATCA,
SEQ ID NO: 83).

### (B)

Schematic of a selected region of the human SMN2 gene that harbors the C-to-T mutation (indicated)

(top strand, 5′-3′ sequence:
TTCCTTTATTTTCCTTACAGGGTTTTAGACAAAATCAAA,
SEQ ID NO: 84).

Potential guide RNA (gRNA) sites targeting this locus with the SpCas9 variant named SpRY are shown, with PAMs and spacer sequences highlighted.

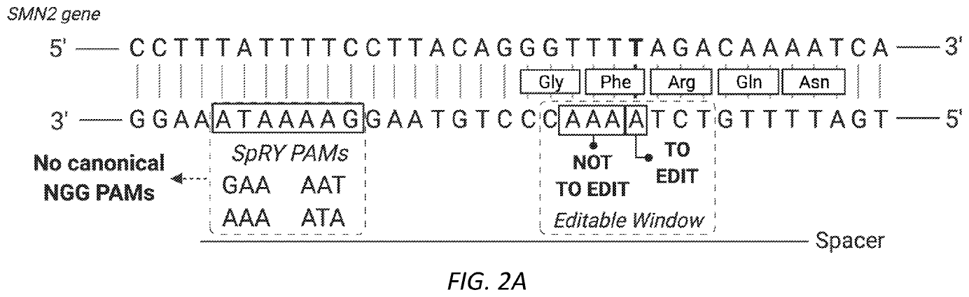

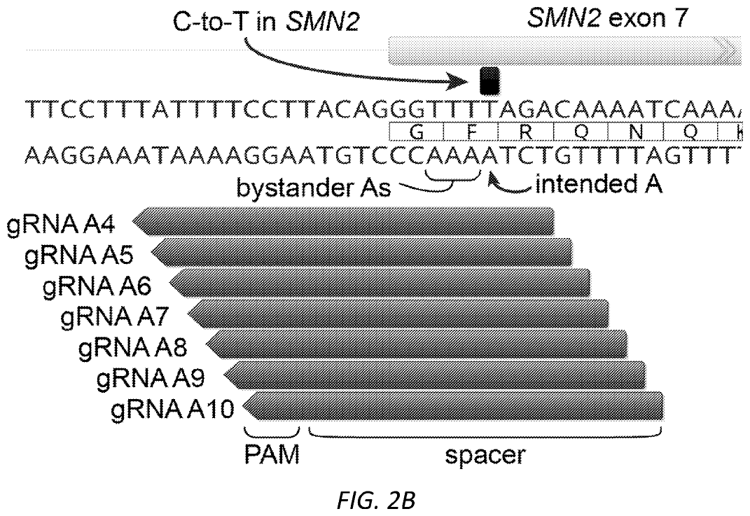

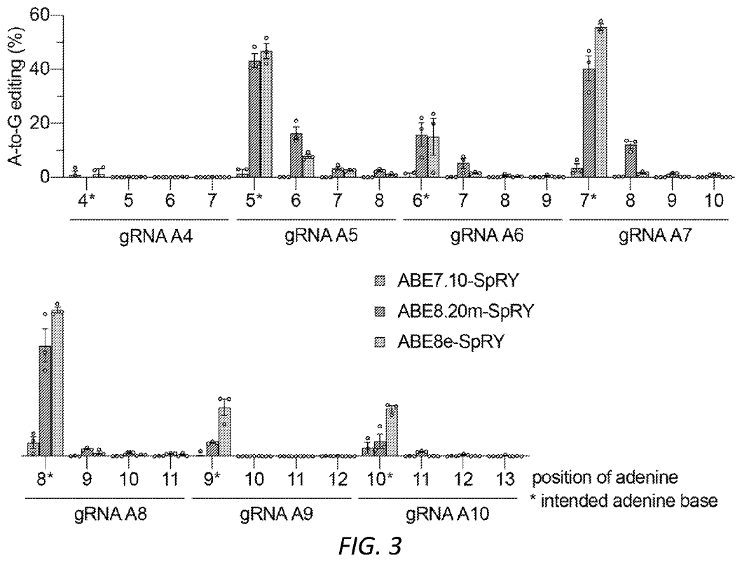

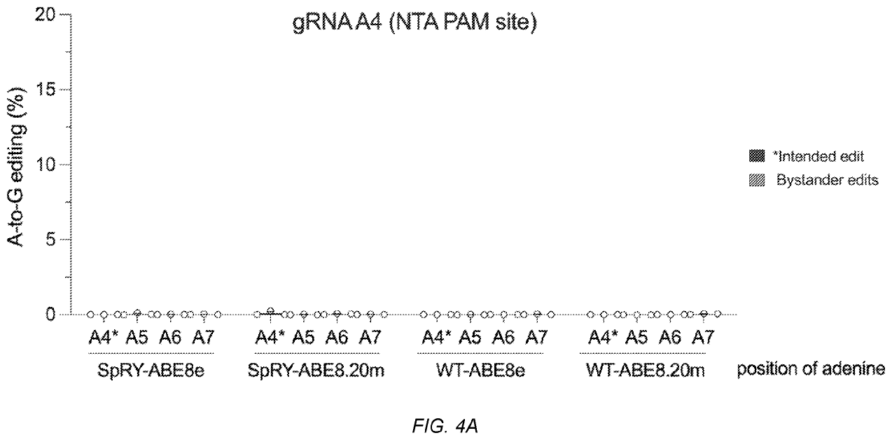

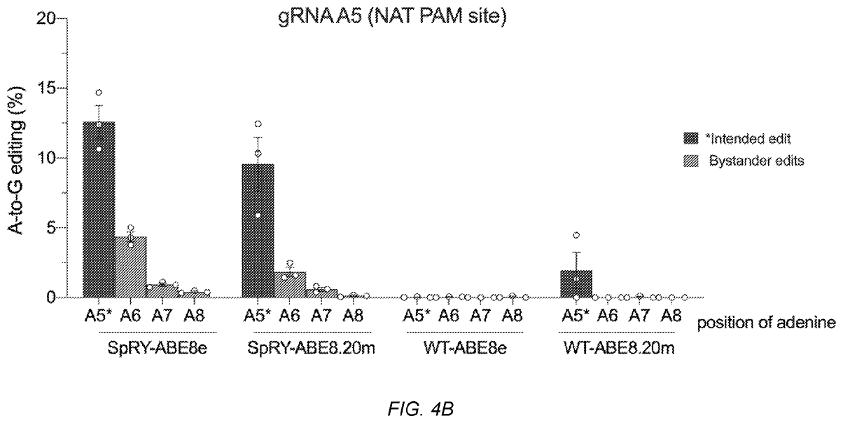

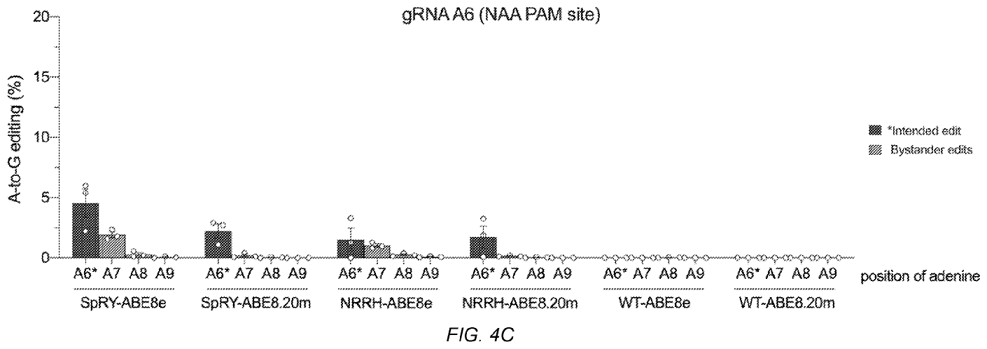

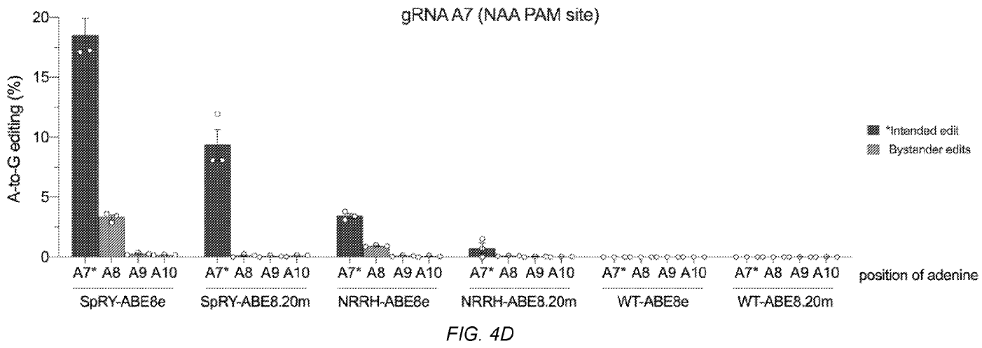

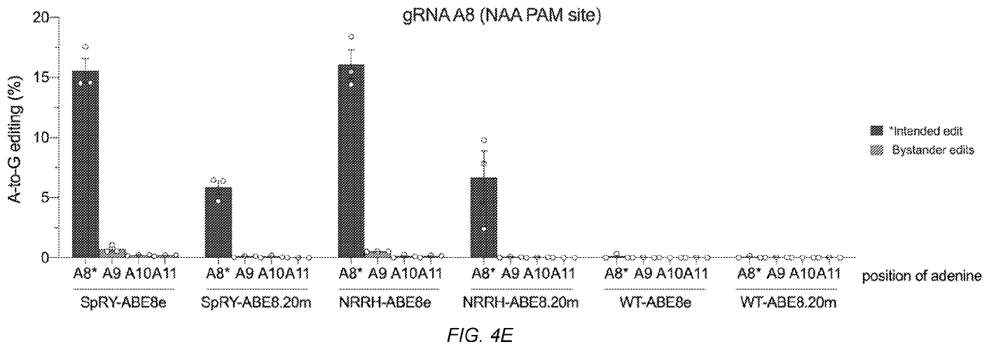

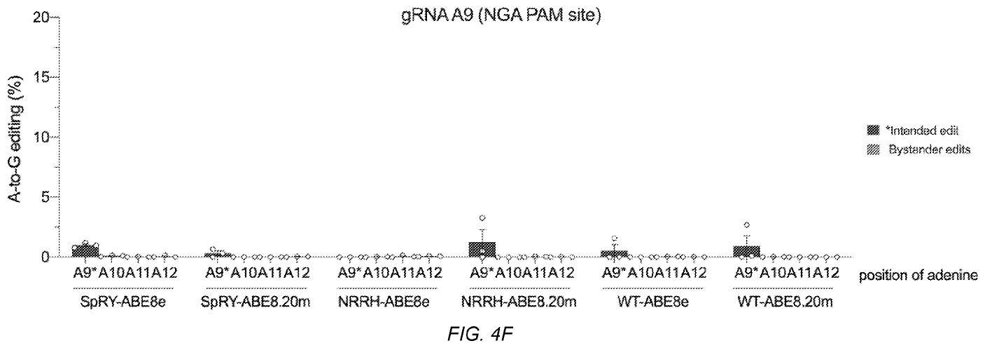

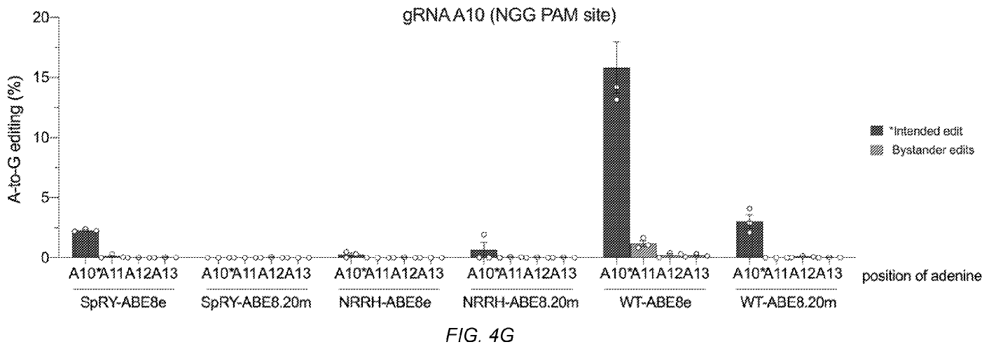

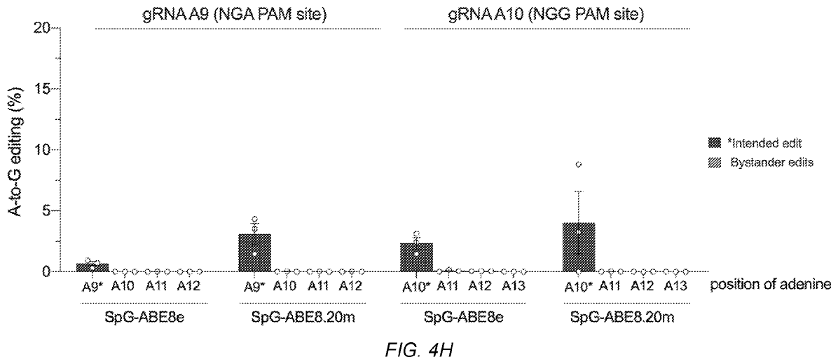

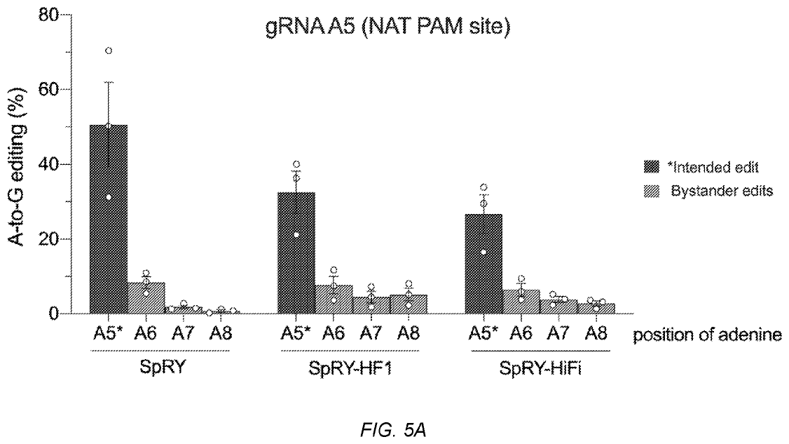

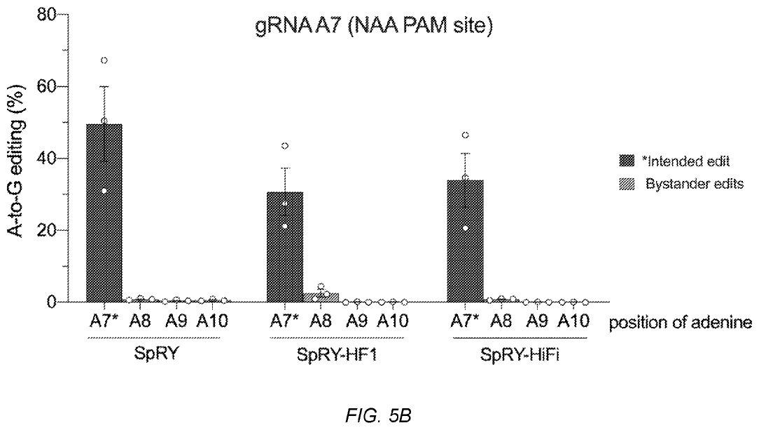

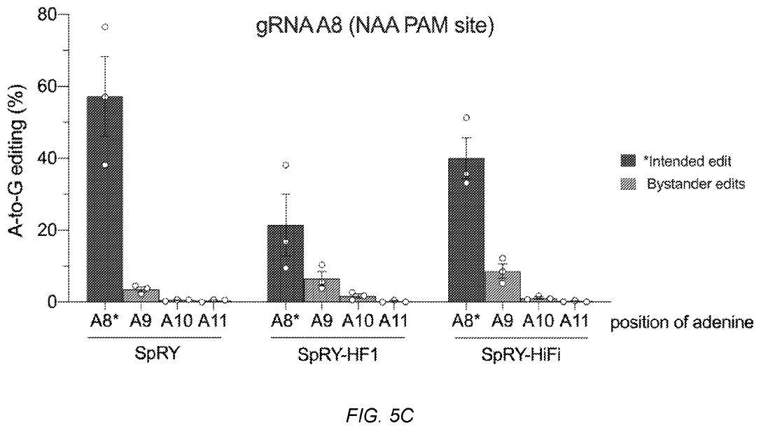

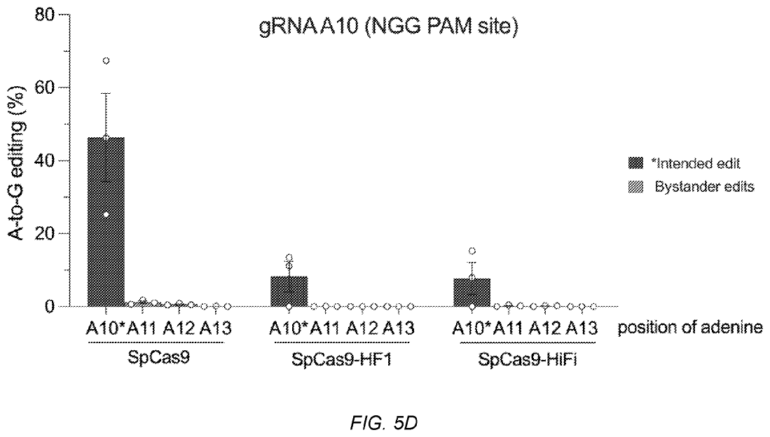

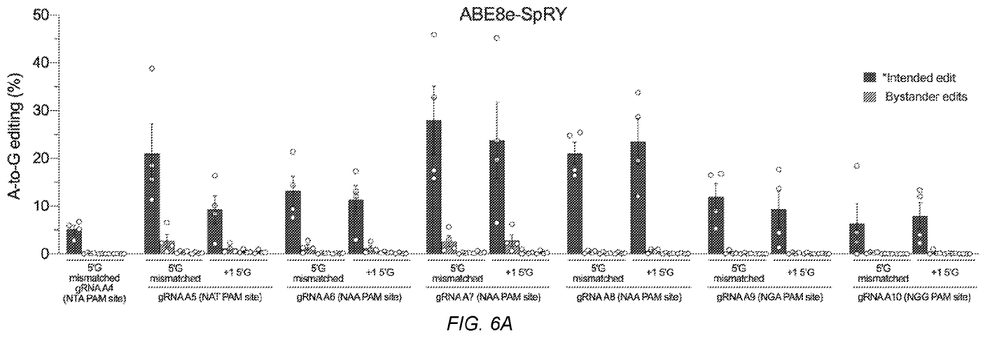

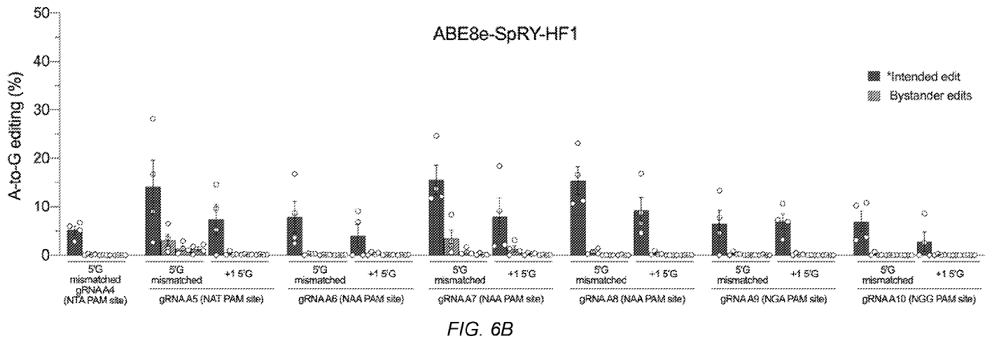

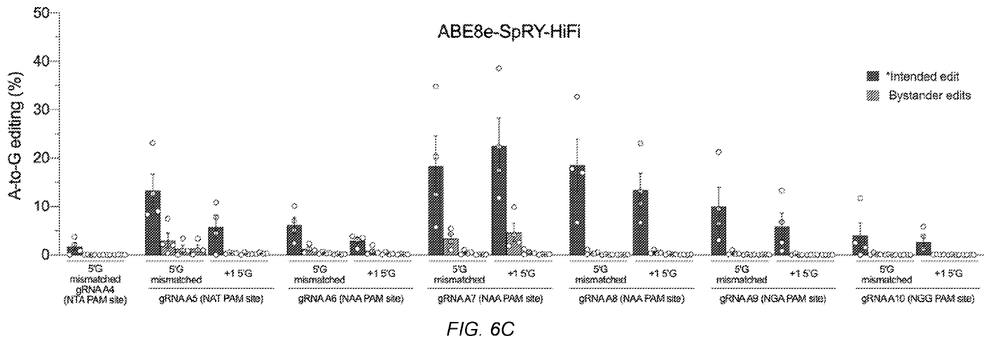

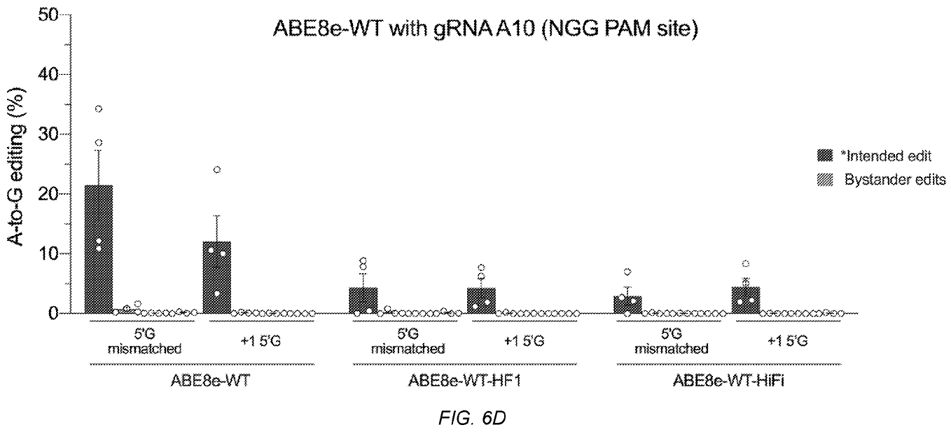

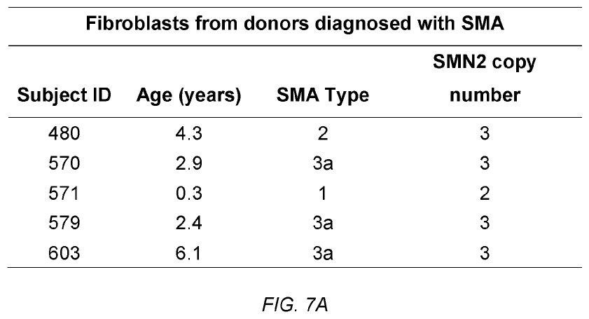

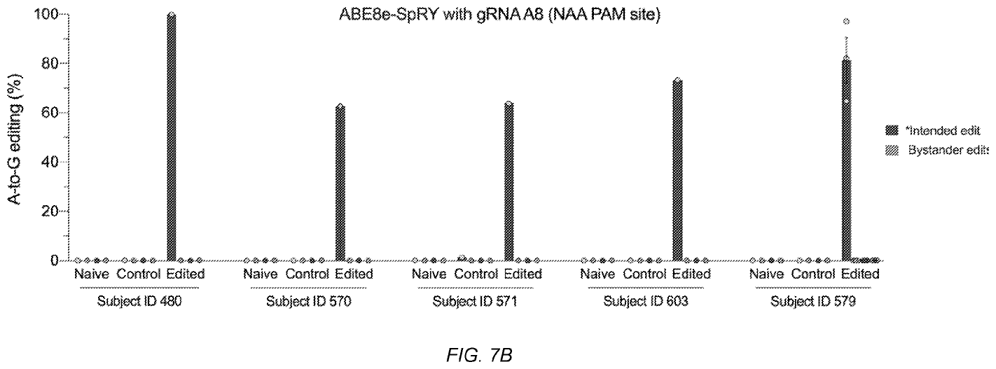

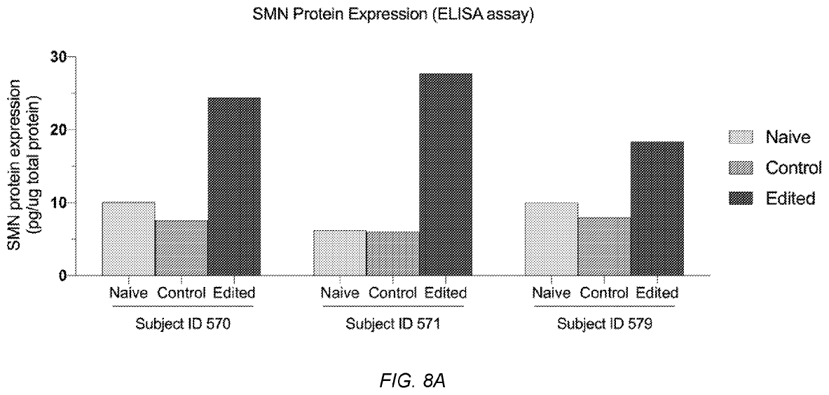

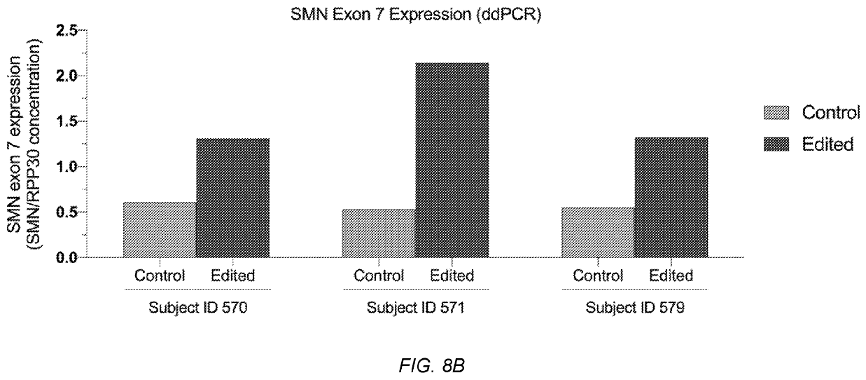

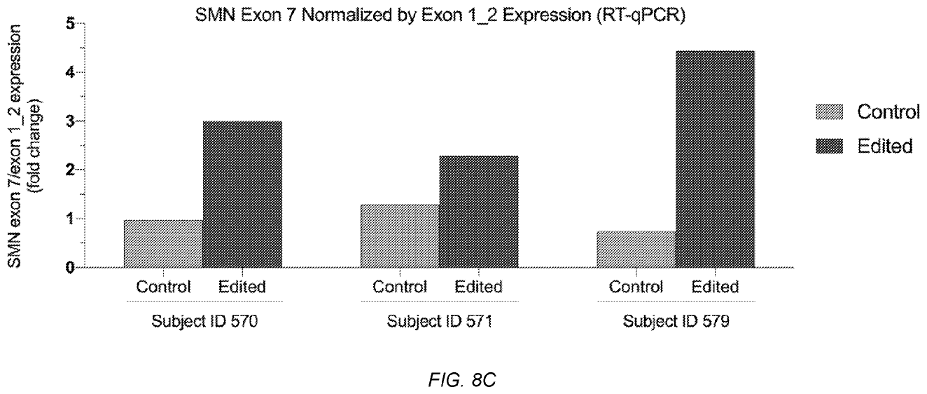

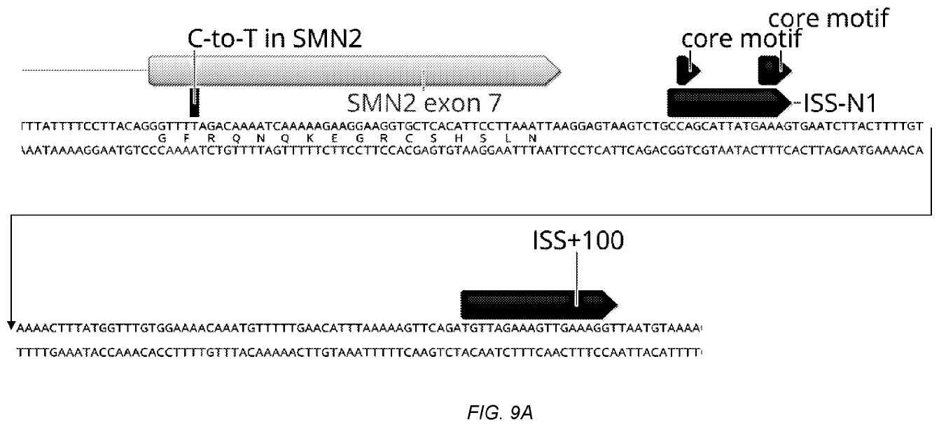

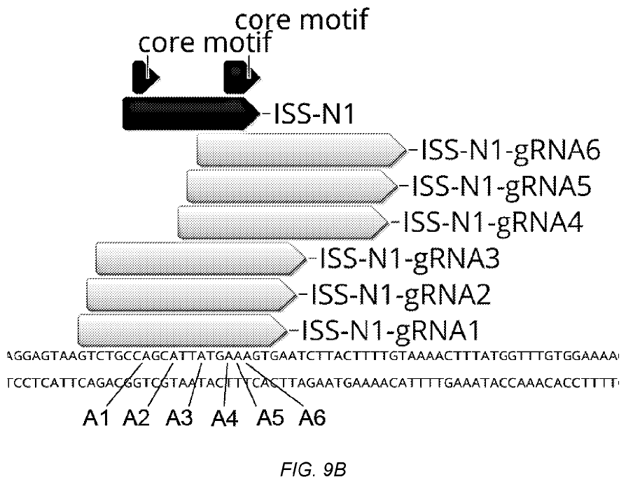

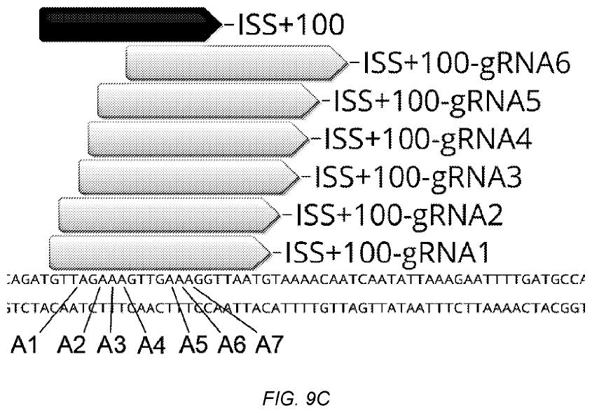

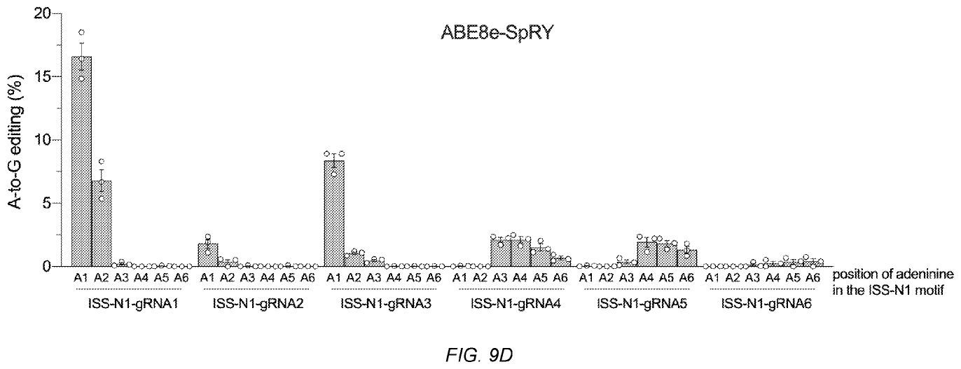

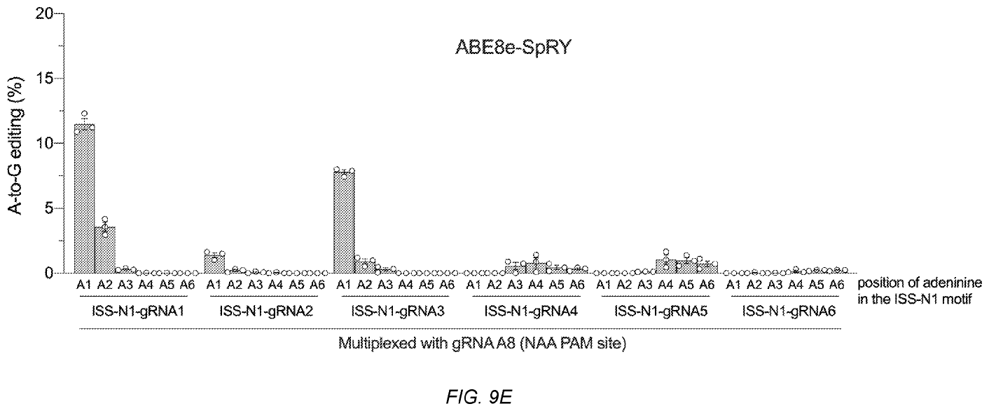

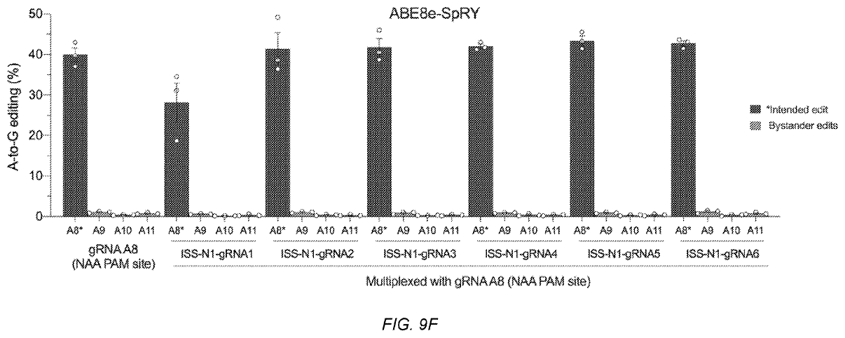

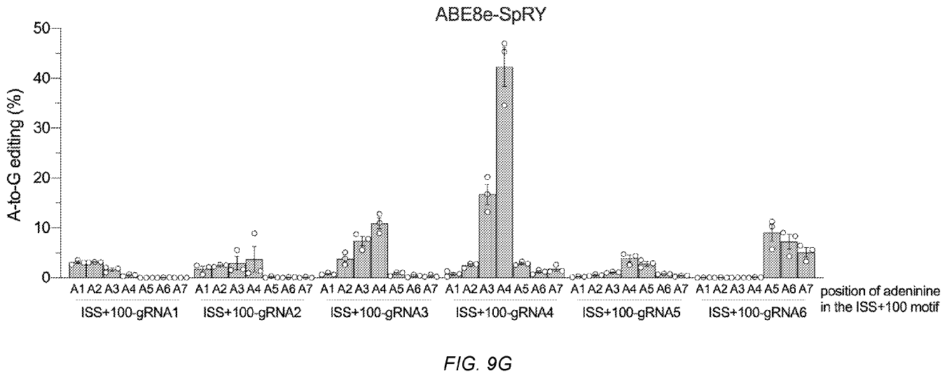

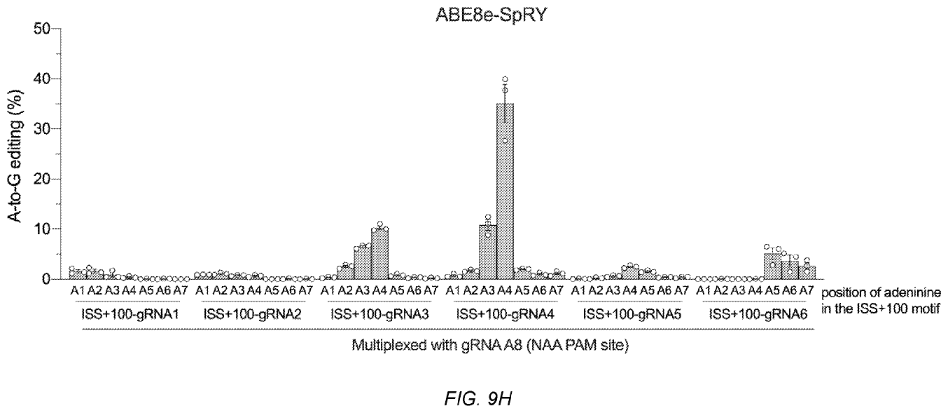

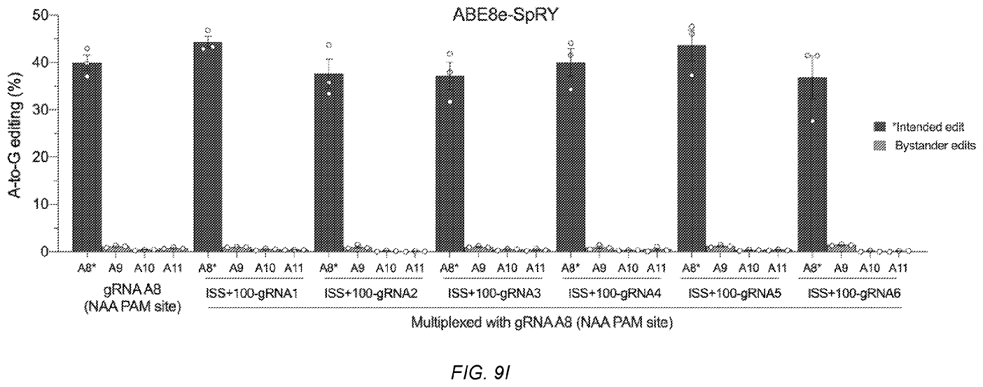

FIG. 3. A-to-G editing of adenine bases in the SMN2 locus (n=3, mean and standard deviation shown). Experiments in human cells were performed using SpRY-ABE constructs and the series of gRNAs shown in FIGS. 2B-C. The position of the intended adenine within the spacer sequence of the gRNA is highlighted for each gRNA using an asterisk (numbering from the PAM-distal end of the spacer).

FIGS. 4A-H. Base editing of SMN2 exon 7 to restore SMN protein expression. (A-H) A-to-G editing in SMN2 exon 7 in HEK 293T cells using gRNAs that positioned the target adenine in position A4 (NTA PAM in panel 4A), A5 (NAT PAM in panel 4B), A6 (NAA PAM in panel 4C), A7 (NAA PAM in panel 4D), A8 (NAA PAM in panel 4E), A9 (NGA PAM in panel 4F), A10 (NGG PAM in panel 4G), and either A9 or A10 when using SpG-ABEs (NGA and NGG PAMs, respectively; panel 4H). Experiments were performed using various ABE constructs, varying the deaminase (ABE8.20m or ABE8e) or SpCas9 PAM variant (wild-type (WT) SpCas9, SpRY, SpCas9-NRRH, or SpG). The position of the intended and bystander adenines within the spacer sequences of each different gRNA are indicated under the x-axis (numbering from the PAM-distal end of the spacer). The target adenine is highlighted for each gRNA using an asterisk; for all experiments, n=3, mean and standard error of the mean (SEM) shown.

FIGS. 5A-D. Base editing of SMN2 exon 7 with high-fidelity ABEs. (A-D) A-to-G editing in SMN2 exon 7 in HEK 293T cells using gRNAs that positioned the target adenine in position A5 (NAT PAM in panel 5A), A7 (NAA PAM in panel 5B), A8 (NAA PAM in panel 5C), and A10 (NGG PAM in panel 5D). Experiments were performed using ABE8e fusions to SpRY, SpRY-HF1, and SpRY-HiFi (panels 5A-5C) or ABE8e fusions to wild-type (WT) SpCas9, SpCas9-HF1, or SpCas9-HiFi (panel 5D). The position of the intended and bystander adenines within the spacer sequences of each different gRNA are indicated under the x-axis (numbering from the PAM-distal end of the spacer). The target adenine is highlighted for each gRNA using an asterisk; for all experiments, n=3, mean and standard error of the mean (SEM) shown.

FIGS. 6A-D. Comparison of gRNA 5′ end architectures for SMN2 exon 7 base editing. (A-D) A-to-G editing in SMN2 exon 7 in HEK 293T cells using gRNAs with either a mismatched 5′G or an additional 21^{st} spacer ‘G’ added (+1 5′G) when targeting various spacers (A5-A10; panels 6A-6C) or just gRNA A10 (panel 6D) with paired with ABE8e-SpRY (panel 6A), ABE8e-SpRY-HF1 (panel 6B), ABE8e-SpRY-HiFi (panel 6C), or with ABE8e fusions to wild-type (WT) SpCas9 (ABE8e-WT), SpCas9-HF1 (ABE8e-WT-HF1), and SpCas9-HiFi (ABE8e-WT-HiFi) (panel 6D). The intended and bystander adenines within the spacer sequences of each gRNA are filled as dark gray or light gray bars, respectively. For all experiments, n=4, mean and standard error of the mean (SEM) shown.

FIGS. 7A-B. Base editing of SMA patient-derived fibroblasts. (A) Information for each of the five SMA patients that underwent skin biopsy to collect fibroblasts. (B) A-to-G editing in SMN2 exon 7 across each of the five SMA fibroblast cell lines transfected with ABE8e-SpRY and gRNA A8. Editing levels in naïve (untransfected), control (transfected with ABE8e-SpRY and a non-SMN2 gRNA) and edited (ABE8e with gRNA A8) samples are shown. For fibroblast lines 480, 570, 571, and 603, a single biological replicate was performed. For fibroblast line 579, n=3 (independent sorting) with mean and standard error of the mean (SEM) shown.

FIGS. 8A-C. Alteration of SMN protein and mRNA levels in edited patient-derived fibroblasts. (A-C) SMA patient-derived fibroblast lines 570, 571, and 579 were transfected with ABE8e-SpRY and gRNA A8 and then subjected to functional assays to assess restoration of SMN expression. Naïve samples were untransfected, Control samples were transfected with ABE8e-SpRY and a non-SMN2 gRNA, and Edited samples were transfected with ABE8e-SpRY and gRNA-A8. (A) SMN protein levels in edited samples, as determined by ELISA. (B) Inclusion of exon 7 into SMN2 mRNA as determined by ddPCR. (C) SMN2 exon 7 mRNA levels relative to the exon 1/2 junction as determined by qPCR. Values for all assays are single biological replicates for each cell line.

FIGS. 9A-I. Base editing of SMN2 intronic splicing silencers to restore SMN protein expression. (A) Schematic of the SMN2 locus that includes exon 7 and the SMN2 intronic splicing silencers (ISSs

(top strand, 5′-3′ sequence:
TTTATTTTCCTTACAGGGTTTTAGACAAAATCAAAAAGAAGGAAGGTGCT
CACATTCCTTAAATTAAGGAGTAAGTCTGCCAGCATTATGAAAGTGAATC
TTACTTTTGTAAAACTTTATGGTTTGTGGAAAACAAATGTTTTTGAACAT
TTAAAAAGTTCAGATGTTAGAAAGTTGAAAGGTTAATGTAAAA,
SEQ ID NO: 85).

The proximity of the target adenine and gRNA targets for SMN2 exon 7 editing relative to the ISSs is highlighted. (B,C) Zoomed-in schematics of the ISS-N1 (panel 9B, top strand, 5′-3′ sequence: AGGAGTAAGTCTGCCAGCATTATG AAAGTGAATCTTACTTTTGTAAAACTTTATGGTTTGTGGAAAA (SEQ ID NO:86) and ISS+100 (panel 9C, top strand, 5′-3′ sequence: CAGATGTTAG AAAGTTGAAAGGTTAATGTAAAACAATCAATATTAAAGAATTTTGATGCCA (SEQ ID NO:87)) regions, with putative gRNAs that could be utilized with A-to-G or C-to-T base editors to disrupt the sequences of these regulatory elements. (D) A-to-G editing of ISS-N1 adenines from experiments performed with ABE8e-SpRY in HEK 293T cells using six gRNAs that target the core ISS-N1 regulatory elements (panel 9D). (E,F) A-to-G editing of ISS-N1 adenines (panel 9E) and SMN2 exon 7 target and bystander adenines (panel 9F) from experiments performed via multiplex gRNA delivery of various ISS-N1 gRNAs paired with SMN2 exon 7 gRNA-A8. (G) A-to-G editing of ISS+100 adenines from experiments performed with ABE8e-SpRY in HEK 293T cells using six gRNAs that target the core ISS+100 regulatory elements (panel 9D). (H,I) A-to-G editing of ISS+100 adenines (panel 9H) and SMN2 exon 7 target and bystander adenines (panel 9I) from experiments performed via multiplex gRNA delivery of various ISS+100 gRNAs paired with SMN2 exon 7 gRNA-A8. For all experiments, n=3, mean and standard error of the mean (SEM) shown.

### DETAILED DESCRIPTION

The primary modifier of severity in spinal muscular atrophy (SMA) is the number of Survival Motor Neuron 2 (SMN2) gene copies. SMN2 is a paralog centromeric gene that differs from SMN1 by a silent C-to-T transition at position 6 in its exon 7, skipping this exon in the majority of mRNA transcripts due to alternative splicing. However, SMN2 gene still produces ˜10% functional SMN protein. Targeting the SMN2 gene with an antisense oligonucleotide or a small molecule to increase the retention of exon 7 and promote a transient increase of full-length SMN protein expression (see, e.g., Hua et al., PLoS Biol. 2007 April; 5(4): e73; Burghes and McGovern, Genes Dev. 2010 Aug. 1; 24(15): 1574-1579) have demonstrated notable clinical results in infants treated early in the disease process (De Vivo et al., Neuromuscul Disord. 2019 November; 29(11):842-856 and Messina and Sframeli, J Clin Med. 2020 July; 9(7): 2222). These therapies include nusinersen (Spinraza, Biogen) and risdaplam (Evrysdi, Roche), both recently approved by the U.S. Food and Drug Administration (FDA). In addition, the FDA also approved onasemnogene abeparvovec (Zolgensma, Novartis), a gene therapy with an adeno-associated virus vector to systemically transduce an SMN1 transgene. Notwithstanding all progress in the field, all available therapies have important limitations. Risdaplam is an oral daily drug administered for patients older than 2 months of age. Hopefully, long term data will validate the efficacy of risdaplam as a medicine to help SMA patients, but this is not a definitive cure for the disease. Nusinersen is delivered intrathecally and is not expected to promote direct effects in non-neuronal cells. Likewise, exogenous SMN1 gene replacement using non-integrating vectors such as onasemnogene abeparvovec presents many challenges, including no efficacy in dividing cells and possible toxic effects of long-term uncontrolled SMN overexpression. Whole blood SMN protein levels correlate with SMN2 copy number in SMA infants, but neither nusinersen nor onasemnogene abeparvovec increase SMN protein levels in whole blood. Identifying additional therapies to permanently replace systemic levels of SMN is highly necessary.

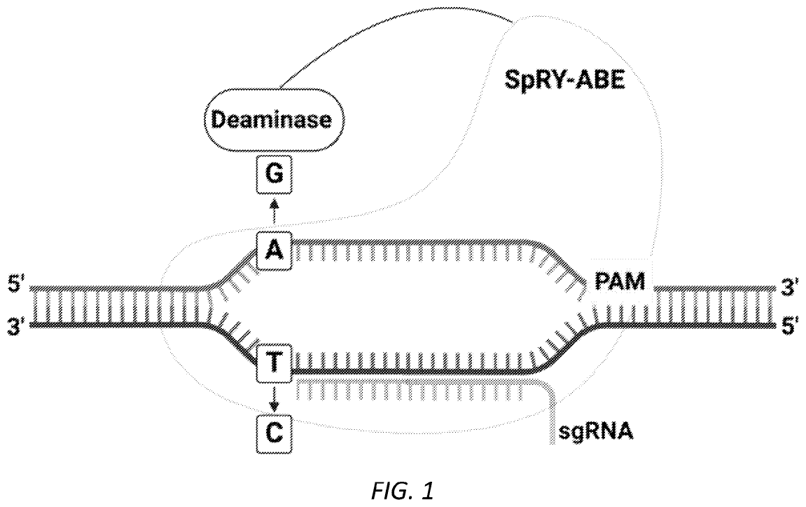

Described herein is a strategy to edit the SMN2 gene to produce full-length SMN protein by applying CRISPR technologies. We leveraged novel variants of Streptococcus pyogenes Cas9 (i.e. SpG and SpRY) (Walton et al., Science. 2020 Apr. 17; 368(6488):290-296) to precisely target previously intractable complex genomic regions in the SMN2 gene. In this case, the Cas9 variant is fused to a base editor deaminase enzyme (FIG. 1). Two main classes of base editors have been developed to date. Cytosine base editors (CBEs) catalyze the conversion of C·G to T·A base pairs using cytidine deaminases, while adenine base editors (ABEs) catalyze A·T to G·C base pairs using an evolved TadA deaminase to convert adenosines to inosines, which are read as guanines by polymerases. It was hypothesized that applying ABEs fused to the novel SpRY variant would result in an efficient tool to convert the adenosine to guanine and, as a consequence, correct the complemented C-to-T transition presented in the exon 7 of SMN2 (FIG. 1, 2A), resulting in higher functional SMN protein levels and potentially a cure for SMA.

The recent development of base editors, including adenine base editors with enhanced activities (e.g., ABE8e (Richter et al., Nature Biotechnology 38:883-891(2020) and ABE8.20-m (Gaudelli et al., Nat. Biotechnol. 2020, 38, 892-900)), allow efficient introduction of single A-to-G nucleotide changes at specified loci in living cells. Thus, the combination of these novel technologies has allowed us to develop an efficient approach to edit the C-to-T transition in the exon 7 of SMN2, which will correct its genetic defect and restore SMN protein expression. In principle, this will create a lasting single-dose cure for SMA.

Provided herein are methods for treating a subject with SMA, to permanently edit the SMN2 gene in cells, e.g., cells of a subject with SMA. The methods include delivering a therapeutically effective amount of the base editors and gRNAs described herein, i.e., an amount sufficient to improve one or more symptoms of the disease or to reduce risk of progression (increased severity) of the disease, e.g., muscle weakness; see US 20200370069 for a description of disease symptoms and clinical classifications. Symptoms can include muscle weakness, poor muscle tone, and (for example, in infants and children) a weak cry, limpness or a tendency to flop, difficulty sucking or swallowing, accumulation of secretions in the lungs or throat, feeding difficulties and increased susceptibility to respiratory tract infections. In some embodiments, the subject is asymptomatic, and the therapeutically effective amount is an amount sufficient to reduce risk of development of symptoms of the disease. Subjects for treatment can be identified by a healthcare provider, e.g., based on presence of a mutation or deletion of the telomeric copy of the gene SMN1 in both chromosomes, which results in the loss of SMN1 gene function. Other diagnostic tests can include electromyography (EMG) to detect reduced muscle electrical activity.

While previous studies have demonstrated that targeting SMN2 is an efficient strategy to increase full-length SMN protein expression, we have designed and applied the most advanced genome editing techniques to permanently edit the SMN2 gene to produce functional SMN protein. In some embodiments, distinguishing features can include:
1. A demonstration, for the first time, of genome editing to correct the C>T mutation in the SMN2 gene that prohibits efficient production of full length SMN protein.
2. The use of novel CRISPR base editors comprised of the newly developed SpCas9 PAM variant SpRY (that allows targeting of previously inaccessible regions of the genome), and new higher activity base editors (e.g., ABE8e). The combination of Cas9s that target variant PAMs (e.g., SpRY) and improved base editors (e.g., ABE8e), enable the generation of the C>T change in SMN2 exon 7.
3. The unexpected finding that when using a particular Cas9 variant, base editor, and guide RNA (gRNA), that we can edit only the intended ‘A’ base that causes the C>T change on the coding strand. Editing this single base requires this particular combination of ABE (SpCas9 PAM variant and adenosine deaminse domain) and gRNA because that ‘A’ base is flanked by 3 other ‘A’s (which presumably would be edited without careful design; the consequence of editing these non-targeted bases is unknown).

### Base Editors

The present methods include contacting a cell, e.g., a cell of a subject who has SMA, with a base editor comprising (i) a Cas9 (which can also be referred to as a Cas9 enzyme or nuclease), wherein the Cas9 is a nickase or catalytically inactive, and (ii) a deaminase domain. Base editors are known in the art, and are described herein. See, e.g., Porto and Komor, Nature Reviews Drug Discovery 19:839-859 (2020); Porto et al., Nature Reviews Drug Discovery 19:839-859 (2020); Sachinidis et al., Signal Transduction and Targeted Therapy 6:221 (2021), doi.org/10.1038/s41392-021-00633-0.

Cas9

The Cas9 portion of the base editor can be, e.g., a “wild type” spCas9 or Cas9 variant that targets NRN or NYN PAMs, e.g., an SpCas9 derivative containing A61R, L1111R, D1135L, S1136W, G1218K, E1219Q, N1317R, A1322R, R1333P, R1335Q, and T1337R substitutions (referred to as SpRY, for SpCas9 variant capable of targeting NRN>NYN PAMs, see Walton et al., Science. 2020 Apr. 17; 368(6488):290-296 and U.S. Ser. No. 62/965,709), SpRY that also comprises HF1 mutations N497A, R661A, Q695A, Q926A (e.g. SpRY-HF1; see Walton et al., Science. 2020 Apr. 17; 368(6488):290-296 and U.S. Ser. No. 62/965,709); SpRY that also comprises a HiFi mutation R691A (e.g. SpRY-HiFi); a Cas9 variant that targets NGN PAMs, e.g., an SpCas9 derivative containing D1135L, S1136W, G1218K, E1219Q, R1335Q, and T1337R substitutions (referred to as SpG, for SpCas9 variant capable of targeting NGN PAMs, see Walton et al., Science. 2020 Apr. 17; 368(6488):290-296 and U.S. Ser. No. 62/965,709); a Cas9 variant that targets NRRH PAMs, e.g., an SpCas9 derivative containing D10T, I322V, 54091, E427G, R654L, R753G, R1114G, D1135N, V1139A, D1180G, E1219V, Q1221H, A1320V, R1333K substitutions or R1114G, D1135N, V1139A, D1180G, E1219V, Q1221H, A1320V, R1333K substitutions (referred to as SpCas9-NRRH, see Miller et. al., Nature Biotechnology. 2020 April;38(4):471-481). The Cas9 must also include a mutation that reduces or abrogates catalytic activity (i.e., a nickase cas9 (nCas9) or dead Cas9 (dCas9, see, e.g., Mali et al., Nat Biotechnol 31, 833-838 (2013); Ran et al., Cell 154, 1380-1389 (2013)), e.g., mutations at D10A to create a single-strand nickase; as used herein, the phrase “wild type” Cas9 refers to the wild type cas9 with at least one mutation that reduces or abrogates catalytic activity. In SpCas9, other mutations that reduce or abrogate catalytic activity are known, e.g., E762A, H840A, N854A, N863A, D986A.

Although herein we refer to Cas9, in general any Cas9-like nuclease could be used based on any ortholog of the Cpf1 protein (including the related Cpf1 enzyme class), including at least one mutation that reduces or abrogates catalytic activity.

TABLE 1
List of Exemplary Cas9 Orthologs

| | UniProt | |
|---|---|---|
| | Accession | Nickase Mutations/ |
| Ortholog | Number | Catalytic residues |
| S. pyogenes Cas9 (SpCas9) | Q99ZW2 | D10A, E762A, H840A, |
| | | N854A, N863A, D986A |
| S. aureus Cas9 (SaCas9) | J7RUAS | D10A and N580A |
| S. thermophilus Cas9 (St1Cas9) | G3ECR1 | D31A and N891A |
| S. pasteurianus Cas9 (SpaCas9) | F5X275 | D10, H599 |
| C. jejuni Cas9 (CjCas9) | Q0P897 | D8A, H559A |
| F. novicida Cas9 (FnCas9) | A0Q5Y3 | D11, N995 |
| F. novicida Cpf1 (FnCpf1) | A0Q7Q2 | D917, E1006, D1255 |
| A. sp. BV3L6 (AsCpf1) | U2UMQ6 | D908, 993E, Q1226, D1263 |

These orthologs, and mutants and variants thereof as known in the art, can be used in any of the fusion proteins described herein. See, e.g., WO 2017/040348 (which describes variants of SaCas9 and SpCas 9 with increased specificity) and WO 2016/141224 (which describes variants of SaCas9 and SpCas 9 with altered PAM specificity).

Deaminase Domain

The deaminase domain can be, e.g., a deaminase domain that modifies adenosine DNA bases, e.g., from adenosine deaminase 1 (ADA1), ADA2; adenosine deaminase acting on RNA 1 (ADAR1), ADAR2, ADAR3; adenosine deaminase acting on tRNA 1 (ADAT1), ADAT2, ADAT3; and naturally occurring or engineered tRNA-specific adenosine deaminase (TadA). Such proteins comprising a base editing domain include cytosine or adenine base editors (CBEs or ABEs), or variants thereof with reduced RNA editing activity, e.g., the SElective Curbing of Unwanted RNA Editing (SECURE)-BE3 variants and SECURE-ABE variants. See, e.g., Gaudelli et al., Nature 551, 464-471 (2017). Grünewald et al., Nature. 2019 May; 569(7756):433-437; Grünewald et al., bioRxiv 631721; doi.org/10.1101/631721; Grünewald et al., Nat Biotechnol. 2019 September;37(9):1041-1048; Abudayyeh et al., Science. 2019 Jul. 26; 365(6451):382-386; and Gehrke et al., Nat Biotechnol. 2018 November; 36(10):977-982. In preferred embodiments, the adenosine deaminase domain is from ABE8e (Richter et al., Nature Biotechnology 38:883-891(2020) or ABE8.20-m (Gaudelli et al., Nat. Biotechnol. 2020, 38, 892-900).

An exemplary sequence of the ABE8e TadA domain is:

(SEQ ID NO: 2)
MSEVEFSHEYWMRHALTLAKRARDEREVPVGAVLVLNNRVIGEGWN
RAIGLHDPTAHAEIMALRQGGLVMQNYRLIDATLYVTFEPCVMCAGAMIH
SRIGRVVFGVRNSKRGAAGSLMNVLNYPGMNHRVEITEGILADECAALLC
DFYRMPRQVFNAQKKAQSSIN.

An exemplary sequence of the ABE8.20m TadA domain is:

(SEQ ID NO: 3)
MSEVEFSHEYWMRHALTLAKRARDEREVPVGAVLVLNNRVIGEGWN
RAIGLHDPTAHAEIMALRQGGLVMQNYRLYDATLYSTFEPCVMCAGAMIH
SRIGRVVFGVRNAKTGAAGSLMDVLHHPGMNHRVEITEGILADECAALL
C.

Linker

In some embodiments, the fusion proteins include a linker between the dCas9 variant and the deaminase domain. Linkers that can be used in these fusion proteins (or between fusion proteins in a concatenated structure) can include any sequence that does not interfere with the function of the fusion proteins. In preferred embodiments, the linkers are short, e.g., 2-20 amino acids, and are typically flexible (i.e., comprising amino acids with a high degree of freedom such as glycine, alanine, and serine). In some embodiments, the linker comprises one or more units consisting of GGGS (SEQ ID NO:69) or GGGGS (SEQ ID NO:70), e.g., two, three, four, or more repeats of the GGGS (SEQ ID NO:69) or GGGGS (SEQ ID NO:70) unit. Other linker sequences can also be used. See, e.g., Kotowski and Sharma, Methods Protoc. 2020 Nov. 18; 3(4):79.

In some embodiments, the fusion protein includes an enzyme, domain, or peptide that inhibits or enhances endogenous DNA repair or base excision repair (BER) pathways, e.g., uracil DNA glycosylase inhibitor (UGI) that inhibits uracil DNA glycosylase (UDG, also known as uracil N-glycosylase, or UNG) mediated excision of uracil to initiate BER; or DNA end-binding proteins such as Gam from the bacteriophage Mu.

The base editors are used in combination with guide RNAs that direct the base editors to deaminate the adenine at position 6 in SMN2 exon 7 (position 6 of CTAAAACCCT (SEQ ID NO:1) and/or to deaminate adenines within the ISS-N1 and ISS+100 motifs in SMN2 intron 7. A number of potential gRNAs are provided herein, e.g., in Table 1. Table 2 provides a list of exemplary preferred combinations of specific base editor variants with specific gRNAs that were identified herein.

TABLE 2
Exemplary base editor variants with best gRNA to edit SMN2 gene

| Base editor | gRNA |
|---|---|
| ABE8e-SpRY, ABE8e-SpRY- | SMN2-ex7-gRNA-A5, SMN2-ex7-gRNA-A5_G, SMN2- |
| HF1, or ABE8e-SpRY-HiFi | ex7-gRNA-A7, SMN2-ex7-gRNA-A7_G, SMN2-ex7- |
| | gRNA-A8, SMN2-ex7-gRNA-A8_G |
| ABE8e-SpCas9 | SMN2-ex7-gRNA-A10 (or SMN2-ex7-gRNA-A10_G) |
| ABE8e-SpRY | ISS-N1-gRNA1, ISS-N1-gRNA3 alone or in |
| | addition to SMN2-ex7-gRNA-A8 |
| ABE8e-SpRY | ISS + 100-gRNA4, ISS + 100-gRNA3, ISS + 100-gRNA7 |
| | alone or in addition to SMN2-ex7-gRNA-A8 |

Methods of Treatment

Provided herein are compositions and methods for treating subjects with SMA; suitable subjects include those diagnosed with SMA, e.g., mammalian and preferably human subjects. The methods include administering to the subject a therapeutically effective amount of the base editors and gRNAs as described herein. A therapeutically effective amount is an amount sufficient to reduce one or more symptoms of SMA in the subject. Symptoms can include muscle weakness and decreased muscle tone; limited mobility; breathing problems; problems eating and swallowing; delayed gross motor skills; spontaneous tongue movements; and scoliosis. Subjects with SMA can be identified by skilled healthcare providers using methods known in the art, including genetic analysis. See, e.g., Keinath et al., Appl Clin Genet. 2021; 14: 11-25.

The base editors and gRNAs can be delivered as proteins or nucleic acids. For example, the base editor and gRNA can be administered as a ribonucleoprotein (RNP) complex (BE protein complexed with gRNA).

Alternatively nucleic acids encoding the base editor and optionally at least one gRNA can be administered. For example, the nucleic acids can include at least one viral vector comprising sequences encoding the base editor and/or gRNA, preferably wherein the viral vector is an AAV. Alternatively, a viral vector encoding the base editor can be administered with a gRNA. Composition comprising (i) mRNA encoding the base editor and (ii) one or more guide RNAs can also be used, preferably wherein the mRNA and gRNA are in lipid nanoparticles (LNPs).

The methods can include administering the proteins, nucleic acids, viral vectors, or compositions using any suitable route, e.g., systemically, optionally intravenously or intraperitoneally, or by intrathecal, intracerebroventricular, intracerebral, or other routes of injection or infusion.

Gene Therapy

In some embodiments, the methods include administering a nucleic acid encoding a base editor, and one or more gRNAs, as described herein to a human subject who has SMA. The nucleic acids can be incorporated into a gene construct to be used as a part of a gene therapy protocol. Expression constructs of such components can be administered in any effective carrier, e.g., any formulation or composition capable of effectively delivering the component gene to cells in vivo. Approaches include insertion of the gene in viral vectors, including recombinant retroviruses, adenovirus, adeno-associated virus, lentivirus, and herpes simplex virus−1, or recombinant bacterial or eukaryotic plasmids. Viral vectors transfect cells directly; plasmid DNA can be delivered naked or with the help of, for example, cationic liposomes (lipofectamine) or derivatized (e.g., antibody conjugated), polylysine conjugates, gramacidin S, artificial viral envelopes or other such intracellular carriers, as well as direct injection of the gene construct or CaPO₄ precipitation carried out in vivo.

A preferred approach for in vivo introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 dl324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986).

Yet another viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immunol.158:97-129 (1992). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). In some embodiments, a rAAV vector comprising an AAV9, AAV9-F, AAVrh10 capsid is used (see Hanlon et al., Mol Ther Methods Clin Dev. 2019 Oct. 23; 15:320-332, WO2020/198737, US2019/0167815, US2020/0370069, and US2020/0360472). In some embodiments, an AAV comprising a capsid protein comprising a targeting sequence is used (see e.g., WO2020014471).

In addition to viral transfer methods, such as those illustrated above, non-viral methods can also be employed to cause expression of a nucleic acid compound described herein (e.g., a base editor and gRNA nucleic acid) in the tissue of a subject. Typically non-viral methods of gene transfer rely on the normal mechanisms used by mammalian cells for the uptake and intracellular transport of macromolecules. In some embodiments, non-viral gene delivery systems can rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al., J. Invest. Dermatol. 116(1):131-135 (2001); Cohen et al., Gene Ther. 7(22):1896-905 (2000); or Tam et al., Gene Ther. 7(21):1867-74 (2000).

In some embodiments, nucleic acids encoding a base editor and gRNA is entrapped in liposomes bearing positive charges on their surface (e.g., lipofectins), which can be tagged with antibodies against cell surface antigens of the target tissue (Mizuno et al., No Shinkei Geka 20:547-551 (1992); PCT publication WO91/06309; Japanese patent application 1047381; and European patent publication EP-A-43075).

In clinical settings, the gene delivery systems for the therapeutic gene can be introduced into a subject by any of a number of methods, each of which is familiar in the art. For instance, a pharmaceutical preparation of the gene delivery system can be introduced systemically, e.g., by intravenous injection, and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the recombinant gene is more limited, with introduction into the subject being quite localized. For example, the gene delivery vehicle can be introduced by catheter (see U.S. Pat. No. 5,328,470) or by stereotactic injection (e.g., Chen et al., PNAS USA 91: 3054-3057 (1994)).

The pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system.

In some embodiments, the vectors include promoters that drive expression in cells and tissues affected by SMA, e.g., spinal cord motor neurons, skeletal muscle, heart and kidney. In some embodiments, the vectors can be delivered systemically or directly to the spinal cord, e.g., by intrathecal or intracerebral injection or infusion. See, e.g., US20190167815, US20200370069 and US20200360472.

See, e.g., Valetdinova et al., Biochemistry (Mosc). 2019 September;84(9):1074-1084; Zhou et al., Hum Gene Ther. 2018 November; 29(11):1252-1263.

Alternatively, a mRNA encoding the base editor can be delivered together with one or more gRNA as described herein. See, e.g., Kenjo et al., Nature Communications 12: 7101 (2021); Qiu et al., PNAS 118 (10) e2020401118 (Mar. 9, 2021); Cheng et al., Nat Nanotechnol. 2020 April; 15(4): 313-320.;

Protein Delivery Systems

To use the base editors described herein to treat subjects with SMA, it may be desirable to deliver them as proteins, e.g., expressed from a nucleic acid that encodes them, in combination with gRNAs. This can be performed in a variety of ways. For example, the nucleic acid encoding the Base editor can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the Base editor for production of the Base editor. The nucleic acid encoding the Base editor can also be cloned into an expression vector, for administration to a plant cell, animal cell, preferably a mammalian cell or a human cell, fungal cell, bacterial cell, or protozoan cell.

To obtain expression, a sequence encoding a Base editor is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

The promoter used to direct expression of a nucleic acid depends on the particular application. For example, a strong constitutive promoter is typically used for expression and purification of fusion proteins. In contrast, when the Base editor is to be administered in vivo for gene regulation, either a constitutive or an inducible promoter can be used, depending on the particular use of the Base editor. In addition, a preferred promoter for administration of the Base editor can be a weak promoter, such as HSV TK or a promoter having similar activity. The promoter can also include elements that are responsive to transactivation, e.g., hypoxia response elements, Gal4 response elements, lac repressor response element, and small molecule control systems such as tetracycline-regulated systems and the RU-486 system (see, e.g., Gossen & Bujard, 1992, Proc. Natl. Acad. Sci. USA, 89:5547; Oligino et al., 1998, Gene Ther., 5:491-496; Wang et al., 1997, Gene Ther., 4:432-441; Neering et al., 1996, Blood, 88:1147-55; and Rendahl et al., 1998, Nat. Biotechnol., 16:757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the Base editor, and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use of the Base editor, e.g., expression in plants, animals, bacteria, fungus, protozoa, etc. Standard bacterial expression vectors include plasmids such as pBR322 based plasmids, pSKF, pET23D, and commercially available tag-fusion expression systems such as GST and LacZ.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

The vectors for expressing the Base editors can include RNA Pol III promoters to drive expression of the guide RNAs, e.g., the H1, U6 or 7SK promoters. These human promoters allow for expression in mammalian cells.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the gRNA encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the Base editor.

Thus, the methods can include delivering the Base editor protein and guide RNA together, e.g., as a complex. For example, the Base editor and gRNA can be can be overexpressed in a host cell and purified, then complexed with the guide RNA (e.g., in a test tube) to form a ribonucleoprotein (RNP), and delivered to cells. In some embodiments, the variant Cas9 can be expressed in and purified from bacteria through the use of bacterial Cas9 expression plasmids. For example, His-tagged variant Cas9 nickases can be expressed in bacterial cells and then purified using nickel affinity chromatography. The use of RNPs circumvents the necessity of delivering plasmid DNAs encoding the nuclease or the guide, or encoding the nuclease as an mRNA. RNP delivery may also improve specificity, presumably because the half-life of the RNP is shorter and there's no persistent expression of the nuclease and guide (as you′d get from a plasmid). The RNPs can be delivered to the cells in vivo or in vitro, e.g., using lipid-mediated transfection or electroporation. See, e.g., Liang et al. “Rapid and highly efficient mammalian cell engineering via Cas9 protein transfection.” Journal of biotechnology 208 (2015): 44-53; Zuris, John A., et al. “Cationic lipid-mediated delivery of proteins enables efficient protein-based genome editing in vitro and in vivo.” Nature biotechnology 33.1 (2015): 73-80; Kim et al. “Highly efficient RNA-guided genome editing in human cells via delivery of purified Cas9 ribonucleoproteins.” Genome research 24.6 (2014): 1012-1019; Wei et al., “Systemic nanoparticle delivery of CRISPR-Cas9 ribonucleoproteins for effective tissue specific genome editing,” Nature Communications 11: 3232 (2020). Nanocarriers, such as liposomes, polymers, and inorganic nanoparticles, can be used for gene delivery. Duan et al., “Nanoparticle Delivery of CRISPR/Cas9 for Genome Editing,” Front. Genet., 12 May 2021| https://doi.org/10.3389/fgene.2021.673286

The present invention includes compositions comprising the Base editors and guide RNAs (e.g., nucleic acids encoding the base editors and gRNAs, or base editor proteins and gRNAs, e.g., RNPs), vectors (e.g., viral expression vectors) expressing the base editors and/or gRNAs, and cells comprising the vectors and optionally expressing the base editors and/or gRNAs.

### EXAMPLES

The invention is further described in the following examples, which do not limit the scope of the invention described in the claims.

### Methods

The following methods were used in the Examples set forth herein.

### Plasmids

The SpRY-ABE human expression plasmids were generated by subcloning either the ABE8e or ABE8.20m open reading frame from a gBlock containing into the Nod and BglII sites of pCMV-T7-ABEmax(7.10)-SpRY-P2A-EGFP (RTW5025; Addgene plasmid 140003). We also similarly generated ABE8e and ABE8.20 versions of wild-type (WT) SpCas9, SpG, and SpCas9-NRRH. The ABE8e-SpRY-HF1 (bearing SpCas9-HF1 mutations N497A/R661A/Q695A/Q926A) and ABE8e-SpRY-HiFi (bearing the SpCas9-HiFi mutation R691A).
Human cell expression plasmids for U6 promoter-driven SpCas9 sgRNAs were generated by annealing and ligating duplexed oligonucleotides corresponding to spacer sequences into BsmBI-digested pUC19-U6-BsmBI_cassette-SpCas9_sgRNA (BPK1520; Addgene plasmid 65777).

### Human Cell Culture

Human HEK 293T cells (ATCC) were cultured in Dulbecco's Modified Eagle Medium (DMEM) supplemented with 10% heat-inactivated FBS (HI-FBS) and 1% penicillin/streptomycin. The supernatant media from cell cultures was analyzed monthly for the presence of mycoplasma using MycoAlert PLUS (Lonza).

### Transfection of Human HEK 293T Cells

All experiments were performed with at least 3 independent biological replicates. For HEK 293T human cell experiments, transfections were performed between 20 and 24 hours following seeding of 2×10⁴ HEK 293T cells per well in 96-well plates. 70 ng of base editor and 30 ng of sgRNA expression plasmids were mixed with 0.72 μL of TransIT-X2 (Mirus) in a total volume of 15 μL Opti-MEM (Thermo Fisher Scientific), incubated for 15 minutes at room temperature, and added to the seeded HEK 293T cells. Experiments were halted after 72 hours. Genomic DNA was collected by discarding the media, resuspending the cells in 100 μL of quick lysis buffer (20 mM Hepes pH 7.5, 100 mM KCl, 5 mM MgCl₂, 5% glycerol, 25 mM DTT, 0.1% Triton X-100, and 60 ng/ul Proteinase K (New England Biolabs; NEB)), heating the lysate for 6 minutes at 65° C., heating at 98° C. for 2 minutes, and then storing at −20° C.

### Assessment of Nuclease Activities in Human Cells

The efficiency of genome modification was determined by next-generation sequencing using a 2-step PCR-based Illumina library construction method. Briefly, genomic loci were amplified from approximately 100 ng of genomic DNA using Q5 High-fidelity DNA Polymerase (NEB). PCR products were purified using paramagnetic beads prepared as previously described. Approximately 20 ng of purified PCR product was used as template for a second PCR to add Illumina barcodes and adapter sequences using Q5 polymerase. PCR products were purified prior to quantification via capillary electrophoresis (Qiagen QIAxcel), normalization, and pooling. Final libraries were quantified by qPCR (Illumina Library qPCR Quantification Kit, KAPA Biosystems) and sequenced on a MiSeq sequencer using a 300-cycle v2 kit (Illumina). Genome editing activities were determined from the sequencing data using CRISPResso2 with the additional command: —min_reads_to_use_region 100.

### Human Fibroblast Culture and Transfection

Fibroblasts were derived from skin biopsies from five different SMA patients. SMA type, SMN2 copy number and age at skin biopsy are provided in FIG. 7A. Fibroblasts were cultured in Dulbecco's Modified Eagle Medium (DMEM) supplemented with 10% heat-inactivated FBS (HI-FBS) and 1% penicillin/streptomycin. The media was modified to contain 20% HI-FBS for recovery after sorting. Fibroblasts were transfected with Lipofectamine LTX (ThermoFisher) to deliver separate plasmids encoding ABE8e-SpRY-P2A-EGFP and SMN2 exon 7 gRNA A8 (or with a non-targeting gRNA to establish a “control” line). Naïve cells were untreated. After 48 hours of transfection, we sorted GFP+ fibroblasts and seeded the pooled GFP+ population to grow for an additional week. Two additional passages were performed to expand the sorted cells, which were then used to extract gDNA, RNA and protein at passage #3.

### SMN Protein Expression

SMN protein levels were measured using an SMN-specific enzyme-linked immunosorbent assay assay (ELISA; Life Sciences Inc., Farmingdale, NY; ADI-900-209) according to the manufacturer's instructions. Sample buffer provided with the ELISA kit was used to extract protein.
qPCR and ddPCR to Measure SMN2 Transcript Levels
RNA was extracted from fibroblasts using the RNeasy Plus Universal Kit (Qiagen, Hilden, Germany). RNA was reverse transcribed using the RT2 First Strand Kit (Qiagen, Hilden, Germany) protocol.
For ddPCR, cDNA was normalized to 2 ng/μL. Each ddPCR reaction contained 12 ng of cDNA, 250 nM each primer, 900 nM probe, and ddPCR supermix for probes (no dUTP) (BioRad), and droplets were generated using a QX200 Automated Droplet Generator (BioRad). PCR products were analyzed using a QX200 Droplet Reader (BioRad) and absolute concentration was determined using QuantaSoft (v1.7.4) and normalized to the level of RPP30 cDNA between samples. The primers and their sequences used for ddPCR include: SMN2 exon 7 forward primer, CAAAAAGAAGGAAGGTGCTCA (SEQ ID NO:71); SMN2 exon 7 reverse primer, TCCAGATCTGTCTGATCGTTTC (SEQ ID NO:72); RPP30 forward primer, GATTTGGACCTGCGAGCG (SEQ ID NO:73); and RPP30 reverse primer, GCGGCTGTCTCCACAAGT (SEQ ID NO:74). The ddPCR probes sequences were TTAAGGAGAAATGCTGGCATAGAGCAGCAC (SEQ ID NO:75, for SMN2-FAM) and TCTGACCTGAAGGCTCTGCGCG (SEQ ID NO:76, for RPP30-HEX). For RT-qPCR, cDNA was normalized to 6.25 ng/μL and cDNA was amplified using SYBR Green (Qiagen, Hilden, Germany) with a Quantstudio 3 real-time PCR system (Applied Biosystems). Each RT-qPCR reaction contained 12.5 ng cDNA, 200 nM each primer, and SYBR green dye (50% of reaction). For each gene, mRNA expression was calculated relative to the average of two housekeeping genes: HPRT and TBP1. The primers and their sequences used for qPCR include: the same SMN2 exon 7 forward and reverse primers described above (i.e., SMN2 exon 7 forward primer, CAAAAAGAAGGAAGGTGCTCA (SEQ ID NO:71); SMN2 exon 7 reverse primer, TCCAGATCTGTCTGATCGTTTC (SEQ ID NO:72)); SMN2 exon 1/2 junction forward primer, ACAACAGTGGAAAGTTGGGGA (SEQ ID NO:77); SMN2 exon 1/2 junction reverse primer, TGAAGCAATGGTAGCTGGGT (SEQ ID NO:78); HPRT forward primer, GAAAAGGACCCCACGAAGTGT (SEQ ID NO:79); HPRT reverse primer, AGTCAAGGGCATATCCTACAA (SEQ ID NO:80); TBP1 forward primer, GCATCACTGTTTCTTGGCGT (SEQ ID NO:81); and TBP1 reverse primer, AGAGCATCTCCAGCACACTC (SEQ ID NO:82).

### Example 1

To treat patients afflicted with SMA, one approach is to correct the silent C-to-T transition in the SMN2 gene that normally prohibits efficient production of the SMN protein. Correction of the C-to-T change would enable full length expression of SMN2, thereby increasing the levels of functional SMN protein in cells. To determine if we could efficiently mediate the conversion of T>C we explored the use of adenine base editors (ABEs). ABEs consist of a catalytically attenuated CRISPR enzyme fused to an adenosine deaminase domain, which permits the installation of A-to-G changes (or T-to-C on the other strand) within the editing window of the deaminase domain.

To edit the SMN2 gene, one must be able to precisely target the ABE construct to properly position the ‘edit window’ of the deaminase domain over the intended base. The restriction of base editors to edit within a small sequence window is compounded by the fact that wild-type CRISPR-Cas9 from Streptococcus pyogenes (SpCas9) requires the recognition of a short NGG protospacer-adjacent motif (PAM) to initiate binding of DNA target sites. When analyzing the sequence in the SMN2 gene near the C-to-T change that causes the defect in SMN protein production, there are no NGG PAMs that would permit targeting this region with wild-type SpCas9. Thus, it is impossible to design optimal gRNAs that would enable base editing utilizing wild-type SpCas9 ABEs (FIG. 1A). Previously, we engineered a novel CRISPR variant, named SpRY, that is no longer constrained to NGG PAMs and can therefore edit freely within the genome. Thus, by using an SpRY-ABE construct, we could precisely target the region of the SMN2 gene required to revert the C-to-T change (by generating a compensatory A-to-G change on the other DNA strand; FIGS. 2A-B).

There are two additional potentially complicating factors for this approach. The first is that the target adenine base in the SMN2 gene is bordered by 3 additional adenines (FIG. 2A). To ensure functional SMN protein is produced without unwanted bystander edits whose consequences are unknown, ideally one would edit only the target adenine without editing the neighboring adenines. Because SpRY can effectively target any sequence, we designed several gRNAs that allow us to ‘tile’ the edit window of the ABE across the target adenine base, placing the target base at all possible positions within the editing window of the deaminase domain. (FIG. 2B) This capability allowed us to avoid bystander edits of the nearby adenine bases. Furthermore, the second complicating factor is that the standard ABE domain, ABE7.10, can sometimes exhibit low levels of A-to-G editing activity. To overcome this constraint, two engineered ABE domains were developed that have improved on-target A-to-G editing efficiencies (named ABE8e and ABE8.20m). We compared the effectiveness of all 3 ABE domains (ABE7.10, ABE8e, and ABE8.20m) to determine which SpRY-ABE domain enabled the highest level of on-target activity, while avoiding unwanted bystander edits. By testing SpRY fusions to all 3 ABE domains, paired with 7 different guide RNAs (gRNAs) that allowed us to tile the target base at different positions of the ABE edit windows, we could examine which combination of Cas9 nickase, ABE domain, and gRNA offered the highest levels of on-target edit with the lowest levels of unwanted bystander edits (FIGS. 1A and 1B).

We examined the levels of A-to-G editing in human cells for all combinations of SpRY-ABEs and tiling gRNAs (FIG. 1B). SpRY-ABE7.10 mediated low levels of A-to-G editing, while SpRY-ABE8.20m and SpRY-ABE8e enabled much higher levels of base editing (where SpRY-ABE8e was generally more effective than SpRY-ABE8.20m). The highest levels of on-target editing activity were observed when using gRNAs that placed the target base in the most optimal position of the predicted edit window of nucleotides 5 through 8, when counting bases from the PAM-distal end of the target site (FIGS. 1A and 1B). Guides placing the target base at position 5, 7 and 8 in the edit window (gRNAs A5, A7, and A8, respectively; see Table 1) had the highest editing activity (A7=A8>A5), either due to optimal positioning of the base in the edit window, strength of the PAM targeted by ABE-SpRY, or the influence of the flanking sequence. Surprisingly, we found that while high levels of A-to-G editing could be achieved at the target base the levels of by-stander edits observed at the 3 neighboring adenine bases was minimal. Notably, when using the gRNA that positions the target base at position 8 of the edit window (gRNA A8), we observed the highest levels A-to-G editing of the intended base with SpRY-ABE8e, with almost undetectable levels of bystander editing (FIG. 1B). Together, this data demonstrates that by using the most optimal combination of Cas9 variant, ABE domain, and gRNA, we could achieve high levels of correction of the mutation in SMN2 that prohibits efficient production of the SMN protein. Correction of this base using SpRY-ABE8e is a promising approach to treat SMA.

### Example 2

Given that we observed high levels of A-to-G editing in SMN2, which should in principle restore expression of SMN2 protein, we further explored the use of ABE8e-SpRY and other Cas9 variants paired with a series of gRNAs (Table 1) to correct SMN2 (FIG. 4). We compared the on-target editing efficiencies of ABE variants (ABE8e¹ and ABE8.20 m²) of SpRY³ and wild-type (WT) SpCas9 when using gRNAs that positioned the target adenine in positions 4-10 of the spacer (A4-10, FIGS. 4A-4G, respectively). These experiments confirmed our prior observations that SpRY-ABEs fused to the ABE8e domain are superior to those fused to ABE8.20m (FIGS. 4A-4G), and also confirmed that gRNAs A5, A7, and A8 led to the highest levels of SMN2 editing with SpRY. Once again, the lowest levels of bystander editing relative to the on-target edit was observed when using gRNA A8. The methods used to assess editing in this experiment did not distinguish editing of SMN2 bystander bases from editing of the analogous region of SMN1 (because the PCR primers used to amplify SMN2 can also amplify SMN1). Since SMN2 and SMN1 are nearly identical at the sequence level, further experiments will be needed to determine whether the ‘bystander SMN2 edits’ are instead on-target edits of the trio of adenines in SMN1 (TTTGTCTGAAACCCTGT). Adenine editing in SMN1 would likely be innocuous in SMA patients, since they generally either null for SMN1 or have a mutated SMN1 gene that does not result in functional SMN expression.

TABLE 1
Sequences of target sites and gRNAs for base
edits within the SMN2 gene

| Tar- | | | | gRNA |
|---|---|---|---|---|
| get | | | | spacer |
| SMN2 | | | | with |
| exon | target site* | | | mismatched |
| 7 | with PAM | spacer only | PAM | 5′G |
| SMN2- | TCTAAAACCCTG | TCTAAAACCCT | ATA | GCTAAAACCC |
| ex7- | TAAGGAAAATA | GTAAGGAAA | | TGTAAGGAAA |
| gRNA- | (SEQ ID | (SEQ ID | | (SEQ ID |
| A4 | NO: 4) | NO: 5) | | NO: 6) |
| SMN2- | GTCTAAAACCCT | GTCTAAAACCC | AAT | GTCTAAAACC |
| ex7- | GTAAGGAAAAT | TGTAAGGAA | | CTGTAAGGAA |
| gRNA- | (SEQ ID | (SEQ ID | | (SEQ ID |
| A5 | NO: 7) | NO: 8) | | NO: 8) |
| SMN2- | TGTCTAAAACCC | TGTCTAAAACC | AAA | GGTCTAAAAC |
| ex7- | TGTAAGGAAAA | CTGTAAGGA | | CCTGTAAGGA |
| gRNA- | (SEQ ID | (SEQ ID | | (SEQ ID |
| A6 | NO: 9) | NO: 10) | | NO: 11) |
| SMN2- | TTGTCTAAAACC | TTGTCTAAAAC | AAA | GTGTCTAAAA |
| ex7- | CTGTAAGGAAA | CCTGTAAGG | | CCCTGTAAGG |
| gRNA- | (SEQ ID | (SEQ ID | | (SEQ ID |
| A7 | NO: 12) | NO: 13) | | NO: 14) |
| SMN2- | TTTGTCTAAAAC | TTTGTCTAAAA | GAA | GTTGTCTAAA |
| ex7- | CCTGTAAGGAA | CCCTGTAAG | | ACCCTGTAAG |
| gRNA- | (SEQ ID | (SEQ ID | | (SEQ ID |
| A8 | NO: 15) | NO: 16) | | NO: 17) |
| SMN2- | TTTTGTCTAAAA | TTTTGTCTAAA | GGA | GTTTGTCTAA |
| ex7- | CCCTGTAAGGA | ACCCTGTAA | | AACCCTGTAA |
| gRNA- | (SEQ ID | (SEQ ID | | (SEQ ID |
| A9 | NO: 18) | NO: 19) | | NO: 20) |
| SMN2- | ATTTTGTCTAAA | ATTTTGTCTAA | AGG | GTTTTGTCTA |
| ex7- | ACCCTGTAAGG | AACCCTGTA | | AAACCCTGTA |
| gRNA- | (SEQ ID | (SEQ ID | | (SEQ ID |
| A10 | NO: 21) | NO: 22) | | NO: 23) |
| Tar- | | | | |
| get | | | | gRNA |
| SMN2 | | | | spacer |
| exon | target site* | | | with +1 |
| 7 | with PAM | spacer only | PAM | 5′G |
| SMN2- | GTCTAAAACCCT | GGTCTAAAACC | AAT | GGTCTAAAACC |
| ex7- | GTAAGGAAAAT | CTGTAAGGAA | | CTGTAAGGAA |
| gRNA- | (SEQ ID | (SEQ ID | | (SEQ ID |
| A5_G | NO: 24) | NO: 25) | | NO: 25) |
| SMN2- | TGTCTAAAACCC | GTGTCTAAAAC | AAA | GTGTCTAAAAC |
| ex7- | TGTAAGGAAAA | CCTGTAAGGA | | CCTGTAAGGA |
| gRNA- | (SEQ ID | (SEQ ID | | (SEQ ID |
| A6_G | NO: 26) | NO: 27) | | NO: 27) |
| SMN2- | TTGTCTAAAACC | GTTGTCTAAAA | AAA | GTTGTCTAAAA |
| ex7- | CTGTAAGGAAA | CCCTGTAAGG | | CCCTGTAAGG |
| gRNA- | (SEQ ID | (SEQ ID | | (SEQ ID |
| A7_G | NO: 28) | NO: 29) | | NO: 29) |
| SMN2- | TTTGTCTAAAAC | GTTTGTCTAAA | GAA | GTTTGTCTAAA |
| ex7- | CCTGTAAGGAA | ACCCTGTAAG | | ACCCTGTAAG |
| gRNA- | (SEQ ID | (SEQ ID | | (SEQ ID |
| A8_G | NO: 30) | NO: 31) | | NO: 31) |
| SMN2- | TTTTGTCTAAAA | GTTTTGTCTAA | GGA | GTTTTGTCTAA |
| ex7- | CCCTGTAAGGA | AACCCTGTAA | | AACCCTGTAA |
| gRNA- | (SEQ ID | (SEQ ID | | (SEQ ID |
| A9_G | NO: 32) | NO: 33) | | NO: 33) |
| SMN2- | ATTTTGTCTAAA | GATTTTGTCTA | AGG | GATTTTGTCTA |
| ex7- | ACCCTGTAAGG | AAACCCTGTA | | AAACCCTGTA |
| gRNA- | (SEQ ID | (SEQ ID | | (SEQ ID |
| A10_G | NO: 34) | NO: 35) | | NO: 35) |
| Tar- | | | | gRNA |
| get | | | | spacer |
| SMN2 | | | | with +1 |
| in- | | | | 5′G |
| tron | target site | | | when |
| 7 | with PAM | spacer only | PAM | needed |
| ISS- | GTCTGCCAGCAT | GTCTGCCAGCA | TGA | GTCTGCCAGCA |
| N1- | TATGAAAGTGA | TTATGAAAG | | TTATGAAAG |
| gRNA1 | (SEQ ID | (SEQ ID | | (SEQ ID |
| | NO: 36) | NO: 37) | | NO: 37) |
| ISS- | TCTGCCAGCATT | TCTGCCAGCAT | GAA | GTCTGCCAGCA |
| N1 | ATGAAAGTGAA | TATGAAAGT | | TTATGAAAGT |
| gRNA2 | (SEQ ID | (SEQ ID | | (SEQ ID |
| | NO: 38) | NO: 39) | | NO: 40) |
| ISS- | CTGCCAGCATTA | CTGCCAGCATT | AAT | GCTGCCAGCAT |
| N1- | TGAAAGTGAAT | ATGAAAGTG | | TATGAAAGTG |
| gRNA3 | (SEQ ID | (SEQ ID | | (SEQ ID |
| | NO: 41) | NO: 42) | | NO: 43) |
| ISS- | TTATGAAAGTGA | TTATGAAAGTG | TTT | GTTATGAAAGT |
| N1- | ATCTTACTTTT | AATCTTACT | | GAATCTTACT |
| gRNA4 | (SEQ ID | (SEQ ID | | (SEQ ID |
| | NO: 44) | NO: 45) | | NO: 46) |
| ISS- | TATGAAAGTGAA | TATGAAAGTGA | TTG | GTATGAAAGTG |
| N1- | TCTTACTTTTG | ATCTTACTT | | AATCTTACTT |
| gRNA5 | (SEQ ID | (SEQ ID | | (SEQ ID |
| | NO: 47) | NO: 48) | | NO: 49) |
| ISS- | ATGAAAGTGAAT | ATGAAAGTGAA | TGT | GATGAAAGTGA |
| N1- | CTTACTTTTGT | TCTTACTTT | | ATCTTACTTT |
| gRNA6 | (SEQ ID | (SEQ ID | | (SEQ ID |
| | NO: 50) | NO: 51) | | NO: 52) |
| ISS + | GTTAGAAAGTTG | GTTAGAAAGTT | ATG | GTTAGAAAGTT |
| 100- | AAAGGTTAATG | GAAAGGTTA | | GAAAGGTTA |
| gRNA1 | (SEQ ID | (SEQ ID | | (SEQ ID |
| | NO: 53) | NO: 54) | | NO: 54) |
| ISS + | TTAGAAAGTTGA | TTAGAAAGTTG | TGT | GTTAGAAAGTT |
| 100- | AAGGTTAATGT | AAAGGTTAA | | GAAAGGTTAA |
| gRNA2 | (SEQ ID | (SEQ ID | | (SEQ ID |
| | NO: 55) | NO: 56) | | NO: 57) |
| ISS + | AGAAAGTTGAAA | AGAAAGTTGAA | TAA | GAGAAAGTTGA |
| 100- | GGTTAATGTAA | AGGTTAATG | | AAGGTTAATG |
| gRNA3 | (SEQ ID | (SEQ ID | | (SEQ ID |
| | NO: 58) | NO: 59) | | NO: 60) |
| ISS + | GAAAGTTGAAAG | GAAAGTTGAAA | AAA | GAAAGTTGAAA |
| 100- | GTTAATGTAAA | GGTTAATGT | | GGTTAATGT |
| gRNA4 | (SEQ ID | (SEQ ID | | (SEQ ID |
| | NO: 61) | NO: 62) | | NO: 63) |
| ISS + | AAAGTTGAAAGG | AAAGTTGAAAG | AAA | GAAAGTTGAAA |
| 100- | TTAATGTAAAA | GTTAATGTA | | GGTTAATGTA |
| gRNA5 | (SEQ ID | (SEQ ID | | (SEQ ID |
| | NO: 64) | NO: 65) | | NO: 66) |
| ISS + | GTTGAAAGGTTA | GTTGAAAGGTT | CAA | GTTGAAAGGTT |
| 100- | ATGTAAAACAA | AATGTAAAA | | AATGTAAAA |
| gRNA6 | (SEQ ID | (SEQ ID | | (SEQ ID |
| | NO: 67) | NO: 68) | | NO: 68) |

*Target base in SMN2 exon 7 shown in bold text (e.g. GTCTAAAAC, SEQ ID NO: 1)

Beyond the use of SpRY ABEs, we also explored editing with other Cas9 proteins. With WT SpCas9 ABEs, for gRNAs A4-A9 we observed very low or no editing of SMN2 (FIGS. 4A-4F), likely due to the requirement for WT SpCas9 to recognize and edit target sites with NGG PAMs. With WT SpCas9 ABEs paired with gRNA A10, which targets a site bearing an NGG PAM, we observed substantial SMN2 editing ABE8e and more modest editing when fused to ABE8.20m (FIG. 4G). We also assessed SMN2 editing when using other previously described PAM variants, including SpG (reference 3) (which can target sites with NGN PAMs) and SpCas9-NRRH (reference 4) (which has been reported to target sites with NRRH PAMs; R is A or G, and H is A, C, or T). With SpG ABEs and gRNA A9 (targeting a site with an NGA PAM), we observed higher levels of editing compared to SpRY ABEs when paired with gRNA A9 (compare FIGS. 4H and 4F), albeit at levels lower than SpRY ABEs using gRNAs A5, A7, or A8. Furthermore, SpG ABEs could also edit SMN2 when using gRNA A10 (NGG PAM; FIG. 4H), although less effectively compared to WT ABEs using the same gRNA (FIG. 4G). Finally, we tested SpCas9-NRRH ABEs when using gRNAS A6-A10 (FIGS. 4C-4G). With gRNAs A6, A7, and A10, we observed inferior editing with SpCas9-NRRH ABEs compared to SpRY; for gRNAs A8 and A9, we observed comparable levels of A-to-G editing between SpRY and SpCas9-NRRH ABEs. Together, these results demonstrate that efficient editing of SMN2 is possible when pairing Cas9 PAM variant ABEs with specific gRNAs.

Next, we explored whether the ABE8e editors were compatible with previously described high-fidelity variants that eliminate or minimize off-target editing. Because SpRY can access more PAMs compared to other more PAM-restricted Cas9 proteins (e.g. WT), SpRY searches a larger fraction of the genome and therefore encounters and potentially edits more off-target sites that bear non-canonical PAMs. We generated ABE8e fusions to SpRY in the presence of HF1 mutations (N497A/R661A/Q695A/Q926A in SpCas9, previously shown to eliminate nearly all off-target editing) or the HiFi mutation 6 (R691A in SpCas9, which can reduce levels of off-target editing). We transfected HEK 293T cells with plasmids encoding ABE8e-SpRY, ABE8e-SpRY-HF1, and ABE8e-SpRY-HiFi when paired with gRNAs A5, A7, and A8 (FIGS. 5A-5C, respectively) and measured on-target editing of the target and bystander adenines within exon 7 of SMN2. Depending on the construct used, the HF1 and HiFi SpRY ABEs retained high levels of SMN2 editing, albeit at reduced efficiencies compared to ABE8e-SpRY (FIGS. 5A-5C), with similar levels of bystander editing (which might also be attributable to off-target editing of the SMN1 gene). When testing SpCas9-HF1 and -HiFi mutations in the context of WT SpCas9 ABE8e and gRNA 10, we observed a more dramatic loss in on-target SMN2 editing (FIG. 5D). It is possible that the high-fidelity mutations exhibit a greater loss in on-target base editing when the target adenine is closer to the boundary of the ABE8e edit window. Collectively, these data reveal that it is possible to edit SMN2 using high-fidelity SpRY ABE constructs.

To determine whether we could further improve the on-target editing efficiency in exon 7 of SMN2, we examined different gRNA architectures that varied in the identity of the 5′ spacer base. gRNA expression is generally regarded as most efficient from a U6 promoter when a G base is present to initiate transcription^{7,8}. Our previous experiments utilized gRNAs with 20 nt spacers that, when necessary, harbored a mismatched 5′ base to a G (Table 1). An additional method to construct gRNAs with 5′ Gs is to add a 5′ G, generating gRNAs with 21 nt spacers⁹. We compared the efficiency of SMN2 base editing when using either the 20 nt spacers with 5′ mismatched Gs or 21 nt spacers with added Gs (+1 5′G; FIGS. 6A-6D). When performing transfections in HEK 293T cells with ABE8e-SpRY, ABE8e-SpRY-HF1, and ABE8e-SpRY-HiFi constructs paired with gRNAs A5-A10 (FIGS. 6A-6C), we observed a range of editing efficiencies depending on the gRNA spacer and 5′ architecture used. In general, we observed comparable performance between the 20 nt 5′ mismatched gRNAs and those with 21 nt+1 5′G spacers for ABE8e-SpRY and ABE8e-SpRY-HiFi (FIGS. 6A and 6C, respectively). However, for SpRY-ABE8e-HF1, the mismatched 5′G gRNAs usually exhibited higher editing compared to the +1 5′G gRNAs (FIG. 6B).

Taken together, this data demonstrates that we can selectively edit the SMN-abrogating adenine of interest in exon 7 of the SMN2 gene, which in principle should restore SMN expression. The levels of editing that we observed using ABE8e-SpRY and gRNA A8 (NAA PAM site) in HEK 293T cells are robust and led to minimal bystander editing of nearby adenine bases. To determine if this SMN2 base editing approach was translatable to SMA patient-derived cells, we extended our experiments into five SMA patient fibroblast cell lines from our local MGH SPOT SMA Longitudinal Population Database Repository (LPDR) database (FIG. 7A). These cell lines have low SMN protein levels when compared to healthy control fibroblasts. Because human SMA fibroblasts are very sensitive to traditional transfection methods, we performed a series of optimization experiments to select the best method for transfection. Briefly, we performed the transfections using Lipofectamine LTX to deliver plasmids encoding ABE8e-SpRY-P2A-EGFP and gRNA A8, and after 72 hours we sorted GFP+ fibroblasts, seeded the pooled GFP+ population to grow for an additional week, and extracted DNA for the next-generation sequencing (NGS). Transfections were performed using the five different SMA fibroblast cell lines; for one of these cell lines (ID 579), we performed three independent biological replicates (including sorting). Remarkably, this strategy resulted in high levels of SMN2 editing across all five SMA cell lines (>60% A-to-G editing of the intended adenine; FIG. 7B). For two lines, we observed complete or nearly complete editing. Importantly, there was no detectable (<1%) bystander editing of adjacent adenines in all transfections (FIG. 7B), either due to reduction of bystander edits in fibroblasts or also the loss of SMN1 in these cell lines. These results demonstrate that our SMN2 ABE8e-SpRY base editing strategy is extensible to, and efficient in, SMA patient derived cells.

To determine if the edited cells exhibit phenotypic increases in SMN transcripts or protein, we selected 3 of these edited cell populations (from line IDs 570, 571 and 579) to perform functional assays. We compared these edited cells to control samples that were either (1) untransfected (“naïve”), or (2) transfected with SpRY-ABE8e and a non-targeting gRNA (“control”). To assess SMN protein levels, we performed an SMN-specific enzyme-linked immunosorbent assay (ELISA) assay. All fibroblast populations edited with ABE8e-SpRY and gRNA A8 exhibited anywhere from a 2- to 4+-fold increase in SMN protein expression when compared with control or naïve cells (FIG. 8A). These data demonstrate for the first time that precise editing of the adenine of interest in exon 7 of the SMN2 gene increased SMN protein levels in SMA patients-derived cells. Next, we evaluated the impact of SMN2 genomic base editing on SMN2 mRNA transcript levels using two methods. First, we compared the inclusion of exon 7 into SMN2 transcripts as assessed by digital droplet PCR (ddPCR). To do so, we extracted RNA from the edited or control cells, generated cDNA, and performed ddPCR using a probe specific to exon 7. We observed a 2- to 3-fold increase in exon 7 retention in the edited cells when compared control treated SMA-fibroblasts (FIG. 8B). Next, we performed qPCR to compare the relative abundance of the exon 7 to the exon 1/2 junction within SMN2 transcripts (FIG. 8C). Consistent with our ddPCR results, we observed a dramatic increase in exon 7 inclusion into SMN2 transcripts (relative to exon 1/2) in the edited SMA-fibroblasts (FIG. 9C), suggesting that base editing promotes increased expression of full-length SMN2 mRNA.

Taken together, these results demonstrate that specific combinations of base editor enzymes (e.g. ABE8e-SpRY) and gRNAs (e.g. SMN2 exon 7 gRNA A8) lead to high levels of SMN exon 7 editing at the genetic level, and also lead to phenotypically relevant increases in SMN protein production and inclusion of exon 7 in SMN2 mRNA. Given that relatively small increases in SMN expression can exhibit a therapeutic benefit in SMA patients, this editing approach forms the basis of a new therapeutic approach to treat SMA.

### Example 3

We also explored whether we could base edit and disrupt other regulatory elements in SMN2, with the goal of restoring SMN protein expression. The C-to-T difference in exon 7 of SMN2 compared to SMN1 causes an approximately 90% reduction in SMN protein expression from SMN2^{10,11}. This single nucleotide change in SMN2 alters splicing regulatory elements for exon 7, leading to exclusion of exon 7 from SMN2 transcripts and thus concomitant reduction in functional SMN protein¹²⁻¹⁴. The mechanisms underlying this alternative splicing involves the disruption of a binding site for pre-mRNA-splicing factors caused by this C-to-T transition^{13,15,16}. Simultaneously, this C-to-T change activates binding sites for the nuclear ribonucleoproteins hnRNPA1 and hnRNPA2 that can repress exon 7 inclusion in the SMN2 mRNA. Interestingly, there are two intronic splicing silencers (ISSs) binding sites for these ribonucleoproteins located in intron 7 of SMN2. It is possible that these de novo ISSs in SMN2 could be edited and disrupted using genome editing technologies. A previous study suggested that nuclease-mediated knockout of two SMN2 ISSs, which lie just downstream of SMN2 exon 7 (FIG. 9A), could enhance SMN protein expression from the SMN2 gene¹⁶. We therefore explored whether we could use our ABE8e-SpRY construct to generate A-to-G edits within the ISSs (rather than nuclease-mediated deletions), an approach that would be compatible with using the ABE8e-SpRY editor to target and correct the C-to-T change in SMN2 exon 7. To do so, we designed six different gRNAs that would position the edit window of the ABE8e-SpRY construct over adenine bases of ISS-N1 and ISS+100 (FIGS. 9B and 9C, respectively).

We transfected HEK 293T cells using ABE8e-SpRY and 12 ISS-targeting gRNAs (6 targeting each of ISS-N1 and ISS+100). For experiments targeting the ISS-N1 motif, we observed editing of several adenines while using various gRNAs (FIG. 9D). The most edited nucleotide was the first adenine in the ISS-N1 motif with >15% A-to-G editing by ABE8e-SpRY when using ISS-N1-gRNA1 (FIG. 9D). This edit would presumably disrupt a core motif if ISS-N1. We then wondered whether we could undertake a multiplex dual editing approach that simultaneously targets these sites and SMN2 exon 7. By transfecting two gRNAs concurrently (one gRNA for the ISS-N1 paired with gRNA A8 for SMN2 exon 7), we found that the same adenines were edited in the ISS-N1 motif (FIG. 9E), with slightly reduced overall editing efficiency when compared to the condition in which only gRNAs designed for the ISS-N1 motif were transfected. For these multiplex edited samples, we also observed robust editing at the target adenine in exon 7 of SMN2 via gRNA A8 (FIG. 9F), suggesting that the dual-editing approach does not appreciably reduce editing of the SMN2 exon 7 target adenine. Next, we performed transfections using the six gRNAs targeted to ISS+100 (FIG. 9C). We observed editing of several adenines within the ISS+100 motif, depending on the gRNA (FIG. 9G). Most notably, with ISS+100-gRNA4, treatment with ABE8e-SpRY led to more than 40% of editing in the fourth adenine of this motif (FIG. 9F). Consistent with the ISS-N1 findings, although editing efficiency within ISS+100 was slightly reduced when co-transfecting with the A8 gRNA for SMN2 exon 7 (FIG. 9H), high levels of A-to-G editing of the target adenine in exon 7 of SMN2 were preserved (FIG. 9I).

### REFERENCES

1. Richter, M. F. et al. Phage-assisted evolution of an adenine base editor with improved Cas domain compatibility and activity. Nat. Biotechnol. 38, 883-891 (2020).
2. Gaudelli, N. M. et al. Directed evolution of adenine base editors with increased activity and therapeutic application. Nat. Biotechnol. 38, 892-900 (2020).
3. Walton, R. T., Christie, K. A., Whittaker, M. N. & Kleinstiver, B. P. Unconstrained genome targeting with near-PAMless engineered CRISPR-Cas9 variants. Science 368, 290-296 (2020).
4. Miller, S. M. et al. Continuous evolution of SpCas9 variants compatible with non-G PAMs. Nat. Biotechnol. 38, 471-481 (2020).
5. Kleinstiver, B. P. et al. High-fidelity CRISPR—Cas9 nucleases with no detectable genome-wide off-target effects. Nature 529, 490-495 (2016).
6. Vakulskas, C. A. et al. A high-fidelity Cas9 mutant delivered as a ribonucleoprotein complex enables efficient gene editing in human hematopoietic stem and progenitor cells. Nat. Med. 24, 1216-1224 (2018).
7. Gao, Z., Harwig, A., Berkhout, B. & Herrera-Carrillo, E. Mutation of nucleotides around the +1 position of type 3 polymerase III promoters: The effect on transcriptional activity and start site usage. Transcription 8, 275-287 (2017).
8. Ma, H. et al. Pol III Promoters to Express Small RNAs: Delineation of Transcription Initiation. Mol. Ther.—Nucleic Acids 3, e161 (2014).
9. Kim, S., Bae, T., Hwang, J. & Kim, J.-S. Rescue of high-specificity Cas9 variants using sgRNAs with matched 5′ nucleotides. Genome Biol. 18, 218 (2017).
10. Lorson, C. L., Hahnen, E., Androphy, E. J. & Wirth, B. A single nucleotide in the SMN gene regulates splicing and is responsible for spinal muscular atrophy. Proc. Natl. Acad. Sci. 96, 6307-6311 (1999).
11. Monani, U. R. et al. A Single Nucleotide Difference That Alters Splicing Patterns Distinguishes the SMA Gene SMN1 From the Copy Gene SMN2. Hum. Mol. Genet. 8, 1177-1183 (1999).
12. Hua, Y., Vickers, T. A., Okunola, H. L., Bennett, C. F. & Krainer, A. R. Antisense Masking of an hnRNP A1/A2 Intronic Splicing Silencer Corrects SMN2 Splicing in Transgenic Mice. Am. J. Hum. Genet. 82, 834-848 (2008).
13. Kashima, T., Rao, N. & Manley, J. L. An intronic element contributes to splicing repression in spinal muscular atrophy. Proc. Natl. Acad. Sci. 104, 3426-3431 (2007).
14. Singh, N. K., Singh, N. N., Androphy, E. J. & Singh, R. N. Splicing of a Critical Exon of Human Survival Motor Neuron is Regulated by a Unique Silencer Element Located in the Last Intron. Mol. Cell. Biol. (2006) doi:10.1128/MCB.26.4.1333-1346.2006.
15. Cartegni, L., Hastings, M. L., Calarco, J. A., de Stanchina, E. & Krainer, A. R. Determinants of Exon 7 Splicing in the Spinal Muscular Atrophy 5 Genes, SMN1 and SMN2. Am. J. Hum. Genet. 78, 63-77 (2006).
16. Li, J.-J. et al. Disruption of splicing-regulatory elements using CRISPR/Cas9 to rescue spinal muscular atrophy in human iPSCs and mice. Natl. Sci. Rev. 7, 92-101 (2020).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a subject who has spinal muscular atrophy (SMA), the method comprising administering to the subject a therapeutically effective amount of:
(i) a base editor comprising a Cas9, wherein the Cas9 is a nickase or catalytically inactive, and a deaminase domain that modifies adenosine DNA bases; and
(ii) a guide RNA (gRNA) that targets the base editor to deaminate the adenine at position 6 in SMN2 exon 7 (position 6 of CTAAAACCCT (SEQ ID NO:1) and/or to deaminate adenines within the ISS-N1 and ISS+100 motifs in SMN2 intron 7.

2. The method of claim 1, wherein the Cas9 is wild-type SpCas9 or a Cas9 variant that targets NGC NGN, NRN, or NYN PAMs.

3. The method of claim 1, wherein the Cas9 variant is an SpCas9 variant that comprises A61R, L1111R, D1135L, S1136W, G1218K, E1219Q, N1317R, A1322R, R1333P, R1335Q, and T1337R substitutions, optionally in combination with mutations N497A, R661A, Q695A, and Q926A, and/or mutation R691A; SpCas9 that comprises D1135L, S1136W, G1218K, E1219Q, R1335Q, and T1337R substitutions; SpCas9 that comprises D10T, I322V, S409I, E427Q R654L, R753Q R1114Q D1135N, V1139A, D1180Q E1219V, Q1221H, A1320V, R1333K substitutions; or SpCas9 that comprises R1114Q D1135N, V1139A, D1180Q E1219V, Q1221H, A1320V, R1333K substitutions.

4. The method of claim 1, wherein the adenosine deaminase domain is from ABE8e or ABE8.20-m.

5. The method of claim 1, wherein the guide RNA comprises a sequence shown in Table 1, preferably SMN2-ex7-gRNA-A5, SMN2-ex7-gRNA-A7, SMN2-ex7-gRNA-A7G, SMN2-ex7-gRNA-A8, SMN2-ex7-gRNA-A8_G, SMN2-ex7-gRNA-A10, SMN2-ex7-gRNA-A10_G, ISS-N1-gRNA1, ISS-N1-gRNA3, ISS+100-gRNA3, ISS+100-gRNA4, or ISS+100-gRNA6.

6. The method of claim 1, comprising administering a base editor and a gRNA as shown in the following table:

| Base editor | gRNA |
|---|---|
| ABE8e-SpRY, ABE8e-SpRY- | SMN2-ex7-gRNA-A5, SMN2-ex7-gRNA-A5_G, SMN2- |
| HF1, or ABE8e-SpRY-HiFi | ex7-gRNA-A7, SMN2-ex7-gRNA-A7_G, SMN2-ex7- |
| | gRNA-A8, SMN2-ex7-gRNA-A8_G |
| ABE8e-SpCas9 | SMN2-ex7-gRNA-A10 (or SMN2-ex7-gRNA-A10_G) |
| ABE8e-SpRY | ISS-N1-gRNA1, ISS-N1-gRNA3 alone or in |
| | addition to SMN2-ex7-gRNA-A8 |
| ABE8e-SpRY | ISS + 100-gRNA4, ISS + 100-gRNA3, ISS + 100-gRNA7 |
| | alone or in addition to SMN2-ex7-gRNA-A8 |

7. The method of claim 1, wherein the base editor and gRNA are administered as a ribonucleoprotein (RNP) complex.

8. The method of claim 7, wherein the RNP complex is administered systemically, optionally intravenously or intraperitoneally, or by intrathecal, intracerebroventricular, intracerebral, or other routes of injection or infusion.

9. The method of claim 1, comprising administering nucleic acids encoding the base editor and at least one gRNA.

10. The method of claim 9, wherein the nucleic acids comprise at least one viral vector comprising sequences encoding the base editor and/or gRNA, preferably wherein the viral vector is an AAV.

11. The method of claim 9, comprising administering a composition comprising (i) mRNA encoding the base editor and (ii) one or more guide RNAs, preferably wherein the mRNA and gRNA are in lipid nanoparticles (LNPs).

12. The method of claim 9, wherein the viral vector or composition is administered systemically, optionally intravenously or intraperitoneally, or by intrathecal, intracerebroventricular, intracerebral, or other routes of injection or infusion.

13-24. (canceled)

25. A composition comprising (i) a base editor comprising a Cas9, wherein the Cas9 is a nickase or catalytically inactive, and a deaminase domain that modifies adenosine DNA bases; and (ii) a guide RNA (gRNA) that targets the base editor to deaminate the adenine at position 6 in SMN2 exon 7 (position 6 of CTAAAACCCT (SEQ ID NO:1) and/or to deaminate adenines within the ISS-N1 and ISS+100 motifs in SMN2 intron 7.

26. The composition of claim 13, wherein the Cas9 is wild-type SpCas9 or a Cas9 variant that targets NGC NGN, NRN, or NYN PAMs.

27. The composition of claim 25, wherein the Cas9 variant is a SpCas9 that comprises A61R, L1111R, D1135L, S1136W, G1218K, E1219Q, N1317R, A1322R, R1333P, R1335Q, and T1337R substitutions, optionally in combination with mutations N497A, R661A, Q695A, and Q926A, and/or mutation R691A; SpCas9 that comprises D1135L, S1136W, G1218K, E1219Q, R1335Q, and T1337R substitutions; SpCas9 that comprises D10T, I322V, S409I, E427Q R654L, R753Q R1114Q D1135N, V1139A, D1180Q E1219V, Q1221H, A1320V, R1333K substitutions; or SpCas9 that comprises R1114Q D1135N, V1139A, D1180Q E1219V, Q1221H, A1320V, R1333K substitutions.

28. The composition of claim 25, wherein the adenosine deaminase domain is from ABE8e or ABE8.20-m.

29. The composition of claim 25, wherein the guide RNA comprises a sequence shown in Table 1, preferably SMN2-ex7-gRNA-A5, SMN2-ex7-gRNA-A7, SMN2-ex7-gRNA-A7_G, SMN2-ex7-gRNA-A8, SMN2-ex7-gRNA-A8_G, SMN2-ex7-gRNA-A10, SMN2-ex7-gRNA-A10_G, ISS-N1-gRNA1, ISS-N1-gRNA3, ISS+100-gRNA3, ISS+100-gRNA4, or ISS+100-gRNA6.

30. The composition of claim 25, comprising a base editor and gRNA as shown in the following table:

| Base editor | gRNA |
|---|---|
| ABE8e-SpRY, ABE8e-SpRY- | SMN2-ex7-gRNA-A5, SMN2-ex7-gRNA-A5_G, SMN2- |
| HF1, or ABE8e-SpRY-HiFi | ex7-gRNA-A7, SMN2-ex7-gRNA-A7_G, SMN2-ex7- |
| | gRNA-A8, SMN2-ex7-gRNA-A8_G |
| ABE8e-SpCas9 | SMN2-ex7-gRNA-A10 (or SMN2-ex7-gRNA-A10_G) |
| ABE8e-SpRY | ISS-N1-gRNA1, ISS-N1-gRNA3 alone or in |
| | addition to SMN2-ex7-gRNA-A8 |
| ABE8e-SpRY | ISS + 100-gRNA4, ISS + 100-gRNA3, ISS + 100-gRNA7 |
| | alone or in addition to SMN2-ex7-gRNA-A8 |

31. The composition of claim 25, wherein the base editor and gRNA are in a ribonucleoprotein (RNP) complex.

32. The composition of claim 25, comprising nucleic acids encoding the base editor and/or gRNA.

33. The composition of claim 32, wherein the nucleic acids comprise at least one viral vector comprising sequences encoding the base editor and/or gRNA, preferably wherein the viral vector is an AAV.

34. The composition of claim 33, comprising (i) mRNA encoding the base editor and (ii) one or more guide RNAs, preferably in lipid nanoparticles (LNPs).

35. The composition of claim 31, wherein the composition is formulated to be administered systemically, optionally intravenously or intraperitoneally, or by intrathecal, intracerebroventricular, intracerebral, or other routes of injection or infusion.


SEQUENCE LISTING (92 sequences)

SEQ ID NO: 1 - DNA, 10 bp, Homo sapiens
ctaaaaccct

SEQ ID NO: 2 - protein, 167 aa, Artificial Sequence
  Description of Artificial Sequence Synthetic polypeptide
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 3 - protein, 146 aa, Artificial Sequence
  Description of Artificial Sequence Synthetic polypeptide
MSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLC

SEQ ID NO: 4 - DNA, 23 bp, Homo sapiens
tctaaaaccctgtaaggaaaata

SEQ ID NO: 5 - DNA, 20 bp, Artificial Sequence
  Description of Artificial Sequence Synthetic oligonucleotide
tctaaaaccctgtaaggaaa

SEQ ID NO: 6 - DNA, 20 bp, Artificial Sequence
  Description of Artificial Sequence Synthetic oligonucleotide
gctaaaaccctgtaaggaaa

SEQ ID NO: 7 - DNA, 23 bp, Homo sapiens
gtctaaaaccctgtaaggaaaat

SEQ ID NO: 8 - DNA, 20 bp, Artificial Sequence
  Description of Artificial Sequence Synthetic oligonucleotide
gtctaaaaccctgtaaggaa

SEQ ID NO: 9 - DNA, 23 bp, Homo sapiens
tgtctaaaaccctgtaaggaaaa

SEQ ID NO: 10 - DNA, 20 bp, Artificial Sequence
  Description of Artificial Sequence Synthetic oligonucleotide
tgtctaaaaccctgtaagga

SEQ ID NO: 11 - DNA, 20 bp, Artificial Sequence
  Description of Artificial Sequence Synthetic oligonucleotide
ggtctaaaaccctgtaagga

SEQ ID NO: 12 - DNA, 23 bp, Homo sapiens
ttgtctaaaaccctgtaaggaaa

SEQ ID NO: 13 - DNA, 20 bp, Artificial Sequence
  Description of Artificial Sequence Synthetic oligonucleotide
ttgtctaaaaccctgtaagg

SEQ ID NO: 14 - DNA, 20 bp, Artificial Sequence
  Description of Artificial Sequence Synthetic oligonucleotide
gtgtctaaaaccctgtaagg

SEQ ID NO: 15 - DNA, 23 bp, Homo sapiens
tttgtctaaaaccctgtaaggaa

SEQ ID NO: 16 - DNA, 20 bp, Artificial Sequence
  Description of Artificial Sequence Synthetic oligonucleotide
tttgtctaaaaccctgtaag

SEQ ID NO: 17 - DNA, 20 bp, Artificial Sequence
  Description of Artificial Sequence Synthetic oligonucleotide
gttgtctaaaaccctgtaag

SEQ ID NO: 18 - DNA, 23 bp, Homo sapiens
ttttgtctaaaaccctgtaagga

SEQ ID NO: 19 - DNA, 20 bp, Artificial Sequence
  Description of Artificial Sequence Synthetic oligonucleotide
ttttgtctaaaaccctgtaa

SEQ ID NO: 20 - DNA, 20 bp, Artificial Sequence
  Description of Artificial Sequence Synthetic oligonucleotide
gtttgtctaaaaccctgtaa

SEQ ID NO: 21 - DNA, 23 bp, Homo sapiens
attttgtctaaaaccctgtaagg

SEQ ID NO: 22 - DNA, 20 bp, Artificial Sequence
  Description of Artificial Sequence Synthetic oligonucleotide
attttgtctaaaaccctgta

SEQ ID NO: 23 - DNA, 20 bp, Artificial Sequence
  Description of Artificial Sequence Synthetic oligonucleotide
gttttgtctaaaaccctgta

SEQ ID NO: 24 - DNA, 23 bp, Homo sapiens
gtctaaaaccctgtaaggaaaat

SEQ ID NO: 25 - DNA, 21 bp, Artificial Sequence
  Description of Artificial Sequence Synthetic oligonucleotide
ggtctaaaaccctgtaaggaa

SEQ ID NO: 26 - DNA, 23 bp, Homo sapiens
tgtctaaaaccctgtaaggaaaa

SEQ ID NO: 27 - DNA, 21 bp, Artificial Sequence
  Description of Artificial Sequence Synthetic oligonucleotide
gtgtctaaaaccctgtaagga

SEQ ID NO: 28 - DNA, 23 bp, Homo sapiens
ttgtctaaaaccctgtaaggaaa

SEQ ID NO: 29 - DNA, 21 bp, Artificial Sequence
  Description of Artificial Sequence Synthetic oligonucleotide
gttgtctaaaaccctgtaagg

SEQ ID NO: 30 - DNA, 23 bp, Homo sapiens
tttgtctaaaaccctgtaaggaa

SEQ ID NO: 31 - DNA, 21 bp, Artificial Sequence
  Description of Artificial Sequence Synthetic oligonucleotide
gtttgtctaaaaccctgtaag

SEQ ID NO: 32 - DNA, 23 bp, Homo sapiens
ttttgtctaaaaccctgtaagga

SEQ ID NO: 33 - DNA, 21 bp, Artificial Sequence
  Description of Artificial Sequence Synthetic oligonucleotide
gttttgtctaaaaccctgtaa

SEQ ID NO: 34 - DNA, 23 bp, Homo sapiens
attttgtctaaaaccctgtaagg

SEQ ID NO: 35 - DNA, 21 bp, Artificial Sequence
  Description of Artificial Sequence Synthetic oligonucleotide
gattttgtctaaaaccctgta

SEQ ID NO: 36 - DNA, 23 bp, Homo sapiens
gtctgccagcattatgaaagtga

SEQ ID NO: 37 - DNA, 20 bp, Artificial Sequence
  Description of Artificial Sequence Synthetic oligonucleotide
gtctgccagcattatgaaag

SEQ ID NO: 38 - DNA, 23 bp, Homo sapiens
tctgccagcattatgaaagtgaa

SEQ ID NO: 39 - DNA, 20 bp, Artificial Sequence
  Description of Artificial Sequence Synthetic oligonucleotide
tctgccagcattatgaaagt

SEQ ID NO: 40 - DNA, 21 bp, Artificial Sequence
  Description of Artificial Sequence Synthetic oligonucleotide
gtctgccagcattatgaaagt

SEQ ID NO: 41 - DNA, 23 bp, Homo sapiens
ctgccagcattatgaaagtgaat

SEQ ID NO: 42 - DNA, 20 bp, Artificial Sequence
  Description of Artificial Sequence Synthetic oligonucleotide
ctgccagcattatgaaagtg

SEQ ID NO: 43 - DNA, 21 bp, Artificial Sequence
  Description of Artificial Sequence Synthetic oligonucleotide
gctgccagcattatgaaagtg

SEQ ID NO: 44 - DNA, 23 bp, Homo sapiens
ttatgaaagtgaatcttactttt

SEQ ID NO: 45 - DNA, 20 bp, Artificial Sequence
  Description of Artificial Sequence Synthetic oligonucleotide
ttatgaaagtgaatcttact

SEQ ID NO: 46 - DNA, 21 bp, Artificial Sequence
  Description of Artificial Sequence Synthetic oligonucleotide
gttatgaaagtgaatcttact

SEQ ID NO: 47 - DNA, 23 bp, Homo sapiens
tatgaaagtgaatcttacttttg

SEQ ID NO: 48 - DNA, 20 bp, Artificial Sequence
  Description of Artificial Sequence Synthetic oligonucleotide
tatgaaagtgaatcttactt

SEQ ID NO: 49 - DNA, 21 bp, Artificial Sequence
  Description of Artificial Sequence Synthetic oligonucleotide
gtatgaaagtgaatcttactt

SEQ ID NO: 50 - DNA, 23 bp, Homo sapiens
atgaaagtgaatcttacttttgt

SEQ ID NO: 51 - DNA, 20 bp, Artificial Sequence
  Description of Artificial Sequence Synthetic oligonucleotide
atgaaagtgaatcttacttt

SEQ ID NO: 52 - DNA, 21 bp, Artificial Sequence
  Description of Artificial Sequence Synthetic oligonucleotide
gatgaaagtgaatcttacttt

SEQ ID NO: 53 - DNA, 23 bp, Homo sapiens
gttagaaagttgaaaggttaatg

SEQ ID NO: 54 - DNA, 20 bp, Artificial Sequence
  Description of Artificial Sequence Synthetic oligonucleotide
gttagaaagttgaaaggtta

SEQ ID NO: 55 - DNA, 23 bp, Homo sapiens
ttagaaagttgaaaggttaatgt

SEQ ID NO: 56 - DNA, 20 bp, Artificial Sequence
  Description of Artificial Sequence Synthetic oligonucleotide
ttagaaagttgaaaggttaa

SEQ ID NO: 57 - DNA, 21 bp, Artificial Sequence
  Description of Artificial Sequence Synthetic oligonucleotide
gttagaaagttgaaaggttaa

SEQ ID NO: 58 - DNA, 23 bp, Homo sapiens
agaaagttgaaaggttaatgtaa

SEQ ID NO: 59 - DNA, 20 bp, Artificial Sequence
  Description of Artificial Sequence Synthetic oligonucleotide
agaaagttgaaaggttaatg

SEQ ID NO: 60 - DNA, 21 bp, Artificial Sequence
  Description of Artificial Sequence Synthetic oligonucleotide
gagaaagttgaaaggttaatg

SEQ ID NO: 61 - DNA, 23 bp, Homo sapiens
gaaagttgaaaggttaatgtaaa

SEQ ID NO: 62 - DNA, 20 bp, Artificial Sequence
  Description of Artificial Sequence Synthetic oligonucleotide
gaaagttgaaaggttaatgt

SEQ ID NO: 63 - DNA, 20 bp, Artificial Sequence
  Description of Artificial Sequence Synthetic oligonucleotide
gaaagttgaaaggttaatgt

SEQ ID NO: 64 - DNA, 23 bp, Homo sapiens
aaagttgaaaggttaatgtaaaa

SEQ ID NO: 65 - DNA, 20 bp, Artificial Sequence
  Description of Artificial Sequence Synthetic oligonucleotide
aaagttgaaaggttaatgta

SEQ ID NO: 66 - DNA, 21 bp, Artificial Sequence
  Description of Artificial Sequence Synthetic oligonucleotide
gaaagttgaaaggttaatgta

SEQ ID NO: 67 - DNA, 23 bp, Homo sapiens
gttgaaaggttaatgtaaaacaa

SEQ ID NO: 68 - DNA, 20 bp, Artificial Sequence
  Description of Artificial Sequence Synthetic oligonucleotide
gttgaaaggttaatgtaaaa

SEQ ID NO: 69 - protein, 4 aa, Artificial Sequence
  Description of Artificial Sequence Synthetic peptide
GGGS

SEQ ID NO: 70 - protein, 5 aa, Artificial Sequence
  Description of Artificial Sequence Synthetic peptide
GGGGS

SEQ ID NO: 71 - DNA, 21 bp, Artificial Sequence
  Description of Artificial Sequence Synthetic primer
caaaaagaaggaaggtgctca

SEQ ID NO: 72 - DNA, 22 bp, Artificial Sequence
  Description of Artificial Sequence Synthetic primer
tccagatctgtctgatcgtttc

SEQ ID NO: 73 - DNA, 18 bp, Artificial Sequence
  Description of Artificial Sequence Synthetic primer
gatttggacctgcgagcg

SEQ ID NO: 74 - DNA, 18 bp, Artificial Sequence
  Description of Artificial Sequence Synthetic primer
gcggctgtctccacaagt

SEQ ID NO: 75 - DNA, 30 bp, Artificial Sequence
  Description of Artificial Sequence Synthetic probe
ttaaggagaaatgctggcatagagcagcac

SEQ ID NO: 76 - DNA, 22 bp, Artificial Sequence
  Description of Artificial Sequence Synthetic probe
tctgacctgaaggctctgcgcg

SEQ ID NO: 77 - DNA, 21 bp, Artificial Sequence
  Description of Artificial Sequence Synthetic primer
acaacagtggaaagttgggga

SEQ ID NO: 78 - DNA, 20 bp, Artificial Sequence
  Description of Artificial Sequence Synthetic primer
tgaagcaatggtagctgggt

SEQ ID NO: 79 - DNA, 21 bp, Artificial Sequence
  Description of Artificial Sequence Synthetic primer
gaaaaggaccccacgaagtgt

SEQ ID NO: 80 - DNA, 21 bp, Artificial Sequence
  Description of Artificial Sequence Synthetic primer
agtcaagggcatatcctacaa

SEQ ID NO: 81 - DNA, 20 bp, Artificial Sequence
  Description of Artificial Sequence Synthetic primer
gcatcactgtttcttggcgt

SEQ ID NO: 82 - DNA, 20 bp, Artificial Sequence
  Description of Artificial Sequence Synthetic primer
agagcatctccagcacactc

SEQ ID NO: 83 - DNA, 35 bp, Homo sapiens
cctttattttccttacagggttttagacaaaatca

SEQ ID NO: 84 - DNA, 39 bp, Homo sapiens
ttcctttattttccttacagggttttagacaaaatcaaa

SEQ ID NO: 85 - DNA, 193 bp, Homo sapiens
tttattttccttacagggttttagacaaaatcaaaaagaaggaaggtgctcacattccttaaattaaggagtaagtctgccagcattatgaaagtgaatcttacttttgtaaaactttatggtttgtggaaaacaaatgtttttgaacatttaaaaagttcagatgttagaaagttgaaaggttaatgtaaaa

SEQ ID NO: 86 - DNA, 67 bp, Homo sapiens
aggagtaagtctgccagcattatgaaagtgaatcttacttttgtaaaactttatggtttgtggaaaa

SEQ ID NO: 87 - DNA, 61 bp, Homo sapiens
cagatgttagaaagttgaaaggttaatgtaaaacaatcaatattaaagaattttgatgcca

SEQ ID NO: 88 - DNA, 9 bp, Homo sapiens
gtctaaaac

SEQ ID NO: 89 - DNA, 17 bp, Homo sapiens
tttgtctgaaaccctgt

SEQ ID NO: 90 - protein, 5 aa, Homo sapiens
GFRQN

SEQ ID NO: 91 - protein, 6 aa, Homo sapiens
GFRQNQ

SEQ ID NO: 92 - protein, 16 aa, Homo sapiens
GFRQNQKEGRCSHSLN